(19) United States
(12) Patent Application Publication
(10) Pub. No.: US 2024/0336979 A1
(43) Pub. Date: Oct. 10, 2024

(54) METHODS OF ANALYSIS OF 17Q22 POLYMORPHISMS ASSOCIATED WITH CANCER AND USES THEREOF

(71) Applicant: UNIVERSIDADE DO ALGARVE, Faro (PT)

(72) Inventors: Joana GONÇALVES DE GOUVEIA MAIA XAVIER, Faro (PT); Ana Teresa LUÍS LOPES MAIA, Faro (PT); Filipa Alexandra OLEIRO ESTEVES RIBEIROS, Faro (PT)

(73) Assignee: UNIVERSIDADE DO ALGARVE, Faro (PT)

(21) Appl. No.: 18/735,246

(22) Filed: Jun. 6, 2024

Related U.S. Application Data
(63) Continuation-in-part of application No. PCT/EP2022/084847, filed on Dec. 7, 2022.

(30) Foreign Application Priority Data
Dec. 7, 2021 (PT) 20211000046950

(51) Int. Cl. C12Q 1/6886; C12Q 1/6851

(52) U.S. Cl. CPC C12Q 1/6886; C12Q 1/6851; C12Q 2600/106; C12Q 2600/156; C12Q 2600/158

(57) ABSTRACT

The invention relates to a method of predicting a subject's likelihood of being diagnosed with a malignant cancer, comprising measuring the relative expression level of a reference allele and an alternative allele of selected cancer risk associated polymorphisms in 17q22 in blood samples.

### CROSS REFERENCE TO RELATED APPLICATIONS

This is a Continuation in Part of International Patent Application No. PCT/EP2022/084847, filed Jun. 7, 2022, which in turn claims the right of priority of Portuguese Patent Application No. 20211000046950, filed Dec. 7, 2021. The contents of the foregoing patent applications are incorporated by reference herein in their entirety.

### FIELD

The present invention relates to a method to quantify a subject's risk of developing cancer, comprising measure the presence of difference in allelic expression of polymorphisms in the 17q22 locus.

### BACKGROUND OF THE INVENTION

Inherited susceptibility to cancer is largely due to polygenic variation. In breast cancer (BC) for example, genome-wide association studies (GWAS) have identified a number of risk-associated loci. Many hundreds of genetic variants linked with cancer are deposited at the NHGRI-EBI GWAS Catalogue (MacArthur et al., 2017 Nucleic Acids Res. 45:D896). Nevertheless, the current knowledge of genetic risk explains only half of all familial breast cancer cases.

A striking observation from GWAS is that most of the loci identified lie outside of genes, in either intergenic regions or the so-called “gene deserts”. Intergenic GWAS loci signals suggest a regulatory, or cis-regulatory role for these risk associated variants, influencing the expression of both close and distant target genes (French et al., 2013 Am. J. Hum. Gen. 92(4):489; Meyer et al., 2011 Plos Gen. 7:e1002165; 2008 Plos Biol. 6:e108). Humans are diploid organisms, and it possible that two alleles of a genetic locus in a heterozygous individual are expressed at a different rate, termed allelic imbalance. Cis-regulatory variation can produce imbalances in the expression of both alleles of autosomal genes, which can be quantified and compared in heterozygous individuals as a ratio of the expression of one allele compared with the other (allelic expression ratio or AE ratio). The difference in allelic expression is henceforth denominated differential allelic expression or DAE (Maia et al., 2009 Breast Can. Re. 11:R88).

Existing biomarker diagnostic assays based on “risk” SNP (rSNP) examine the DNA of a subject and determine whether they are wildtype, heterozygous, or homozygous for a risk-associated variant sequence, then assign the patient a risk status according to the “dose” (number of risk alleles) of the alternative sequence in their DNA. Such assays can identify individuals who have, or are likely to develop a Mendelian disease (a disease which occurs a single allele bears a specific mutation, such as Tay-Sachs disease, or cystic fibrosis), and individuals who bear highly penetrative mutations associated with a very high risk of developing certain types of cancer, such as the BRCA1 and BRCA2 mutations, where an increase in mutant allele dose is strongly associated with being diagnosed with cancer. They are less useful at predicting the risk of developing diseases with a more complex etiology. A small number of studies have examined the DAE for candidate genes using in loci associated with susceptibility to complex diseases, including colorectal cancer (Valle et al., 2008 Science 321:1361). Compounding this problem, most risk SNP discovered by GWAS studies are in non-coding regions, meaning they are not amenable to allele-specific transcription measurements. Their impact on cancer development is due to cis-regulatory activity on target genes, whose expression is not similarly associated with risk in even highly powered GWAS studies, due to the impact of complicated networks of regulation and the presence of multiple SNPs which are often found together, making it difficult to determine which is most predictive.

Other diagnostic models combine DNA genotyping of multiple alleles associated with risk, in order to provide a more accurate prediction of risk for an individual based on the impact of multiple SNP/genes associated with disease. However, for most “risk” SNPs, subjects develop disease much more rarely, meaning they are not useful in a genetic counselling context.

For the reasons above, current state of the art genetic diagnostic methods which rely on DNA sequencing fail to capture the impact on gene transcription of a risk locus, and the downstream effect this has on a subject's likelihood of developing disease.

Based on the above-mentioned state of the art, the objective of the present invention is to provide means and methods to predict genetic predisposition to disease susceptibility for the purpose of genetic screening, providing a more accurate quantification of the risk of a heritable disease in an individual than current methodology. This objective is attained by the subject-matter of the independent claims of the present specification, with further advantageous embodiments described in the dependent claims, examples, figures and general description of this specification.

### SUMMARY OF THE INVENTION

By studying genetic variants in the 17q22 locus associated with several forms of cancer, the inventors noted that differential allelic imbalance, in other words, unbalanced overexpression of one allele in patient heterozygous for a risk associated gene variant (or a single nucleotide polymorphism (SNP) in strong linkage disequilibrium with a risk associated gene variant) more accurately predicts disease than the presence of the risk associated gene variant alone. In addition, for some risk associated SNP, they observed overexpression of the wildtype locus, and not the risk associated gene variant was unexpectedly correlated with disease incidence. The inventors applied these findings to develop a biomarker assay for a disease risk locus identified by GWAS, measuring both the amount, and direction of DAE to improve predictive outcomes. (1) Target genes under the control of cis-regulatory variants were first identified for a given locus via differential allele-specific expression (DAE) studies; (2) causal variants associated with DAE at the disease risk locus were mapped; and (3) significant differences in gene expression regulation between patients and healthy individuals were used to derive a threshold for hereditary gene risk, applied in a genetic screening assay for the hereditary disease.

A first aspect of the invention relates to a method for determining the whether a patient has an above average risk of developing a hereditary cancer disease. Risk SNP according to the invention are more frequently present in subjects having been diagnosed with the hereditary cancer disease in the general, and express DAE of either a wildtype (reference) allele, or a variant (alternative allele).

The method comprises the following steps.
obtaining a tissue sample from a genetic screening subject;
at one or more risk single nucleotide polymorphism (SNP) sites, measuring the expression level of:
a reference, or wildtype allele, and
if present, an alternative, or variant allele;
assigning the subject an above average risk of developing said hereditary cancer disease, if any one of the one or more risk SNPs analyzed in the sample is found to be characterized by:
heterozygosity at one or more risk SNP, (both a wildtype and an alternative allele are present), and
DAE in a direction previously determined to be associated with cancer patients is observed, i.e. the ratio of the expression level of the alternative allele, or the expression level of the reference allele is above, or below a threshold for DAE; or
assigning a no more than average risk of developing the hereditary cancer disease to the subject, if for each of the one or more risk SNP:
if the wildtype allele and the alternative allele are expressed at a similar level (i.e. if no DAE is observed),
DAE in the opposite direction previously determined to be associated with cancer patients is observed (i.e. when the allelic expression ratio does not meet a threshold which distinguishes DAE in patient samples from control samples in a test cohort), or
no alternative allele is present at any of the one or more risk SNP.

In particular embodiments, the presence of the risk SNP is significantly associated with the hereditary cancer disease in a GWAS or large-scale genetic study. In other embodiments one or more of the risk SNP is not itself associated with a risk of developing a hereditary cancer disease in such a study, but is a SNP in linkage disequilibrium with a risk SNP identified in a GWAS or large-scale genetic study as above.

In particular embodiments, one or more of the risk SNP is located in a transcribed region of the genes STXBP4, COX11 and/or TOM1L1. In more particular embodiments, the one or more risk SNP is selected from the risk SNP rs17817901, rs12936860, or rs2628315.

In particular embodiments, the sample is a blood sample. In other embodiments, the sample is breast tissue. In particular embodiments the method is used to determine whether a subject has an above average risk of developing ovarian cancer, prostate cancer, breast cancer or lung cancer.

In particular embodiments, the expression level of the wildtype and variant allele present at the risk SNP are compared to a threshold value. The threshold distinguishes equivalent expression of two alleles in a heterozygous individual, from non-equivalent, DAE. In more particular embodiments, the threshold is derived from comparing the allelic expression of a cohort of samples from patients with the hereditary cancer disease to healthy controls, using a normalised measure of effect size, for example a Hedge's g test. Healthy controls refers to a subject who has not been diagnosed with a malignant cancer.

In certain embodiments, the expression level of each allele present at a risk SNP is measured by SNP-sensitive primers using a real time quantitative polymerase chain reaction assay, mRNA sequencing, or by microarray.

### BRIEF DESCRIPTION OF THE DRAWINGS AND TABLES

FIG. 1 shows that genes in the 17q22 locus are under the effect of cis-regulatory variants genetically related to breast cancer risk variants. Boxplots of the allelic expression (AE) ratios for four variants in strong LD with lead risk-SNP rs2787486, located in the genes indicated above the graph. One-sample t-test for mean allelic expression equal to zero: *P<10⁻²; **P<10⁻⁵; ***P<10⁻¹⁰; Boxplots and data points are colored based on LD with rs2787486: yellow r²≥0.6, orange r²≥0.7, and red r²=1.

FIG. 2 shows case-control study using allelic expression ratios identifies risk in the 17q22 locus in breast tissue and blood samples. Cumming estimation plot of Hedges' g between breast cancer cases and controls for allelic expression ratios calculated at rs17817901 (ratio calculated as allele G by allele A) and rs2628315 (ratio calculated as allele A by allele G) in normal breast and blood. The heterozygous individuals for each indicated variant and tissue are plotted on the upper axes. The vertical lines next to the raw data correspond to the conventional mean±standard deviation error bars, where the mean of each group is indicated as a gap in the line. The mean difference is plotted on the lower axes as a bootstrap sampling distribution (bootstrap n=5000). The mean differences are depicted as dots, and the 95% confidence intervals are indicated by the ends of the vertical error bars.

FIG. 3 Case-control study using allelic expression ratios identifies risk in the CDC16 locus in breast tissue samples. Cumming estimation plot of Hedges' g between breast cancer cases and controls for allelic expression ratios calculated at and rs3211416 (ratio calculated as allele A by allele G) in normal breast. The mean difference in AE ratios is plotted on the lower axes as a bootstrap sampling distribution (bootstrap n=5000). The mean differences are depicted as dots, and the 95% confidence intervals are indicated by the ends of the vertical error bars.

Table 1 shows linkage disequilibrium and DAE for 24 candidate DAE in the 17q22 locus. MAF—alternative allele frequency; p.adjust—p-value adjusted for multiple testing with Bonferroni method; CIinf and CIsup—Inferior and superior limits of the 95% confidence interval (CI) of the mean.

Table 2 Association between AE ratios at two variants in 17q22 and Breast Cancer Risk statistics n—number of samples; CIinf and CIsup—Inferior and superior limits of the 95% confidence interval (CI) of the Hedges' g; p.perm—Permuted Mann-Whitney test p-values.

Table 3 Chromosome position of SNP evaluated in this study, and penetrance of the variant alleles at the population level. Chr—chromosome; pos_hg38—genomic position according to hg38; rsID—variant ID in dbSNP build 141; alt—alternative allele; AFR, AMR, ASN, EUR—minor allele frequency in continental populations (AFR, AMR, ASN, EUR).

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbour Laboratory Press, Cold Spring Harbour, N.Y. and Ausubel et al., Short Protocols in Molecular Biology (2002) 5th Ed, John Wiley & Sons, Inc.) and chemical methods.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

### General Molecular Biology: Nucleic Acid Sequences, Expression

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The term STXBP4 in the context of the present specification relates to the human gene Syntaxin Binding Protein 4 (ENSG00000166263), sometimes referred to as Synip or MGC50337.

The term COX11 in the context of the present specification relates to the human gene cytochrome c oxidase copper chaperone COX11 (ENSG00000166260).

The term TOM1L1 in the context of the present specification relates to the human gene target of myb1 like membrane trafficking proteins (ENSG00000141198).

The term genotype in the context of the present specification relates to the copy number of either a wildtype, also referred to as a reference allele, or an alternative allele, sometimes referred to as a mutant, or variant allele, present for a specific gene or genetic locus. In other words, whether a human subject is heterozygous or homozygous for a particular nucleic acid sequence, for example a risk SNP located in the 17q22 region of chromosome 17. A heterozygous subject, has two different alleles, one copy inherited from each parent. For the risk SNP described herein, the vast majority of patients and healthy subjects are heterozygous, expressing both a reference allele, and an alternative allele.

The term allele, in the context of the present specification relates the two different copies of a genetic locus present in the genome. As diploid organisms, humans have two alleles at most locations, one inherited from each parent. Somatic mutations giving rise to a variant allele may also arise later in life. Heterozygous COX11 and STXBP4 variant alleles for example, are those wherein in one individual, one copy of the genetic locus is characterized by a reference allele, referring to a nucleotide base, or gene sequence found in a reference genome, that differs from an alternative allele, referring to any base other than the reference. Such variant alleles are particularly useful sites at which to measure differential gene expression according to the methods according to the invention. For the risk SNP described herein, the allele matching the reference genome GRCh38.p13 was assigned as the reference allele according to the invention, from which classification thresholds and directions of AE expression were obtained.

The term Single nucleotide polymorphisms (SNP) in the context of the present specification relates to variant alleles corresponding to a single nucleotide position. For example, a single nucleotide position of the reference genome that has one or more alternative nucleotides present in the human population. The reference allele is defined as the nucleotide found in the reference genome, while an alternative allele is any other nucleotide described at the same position.

The terms gene expression or expression, or alternatively the term gene product, may refer to either of, or both of, the processes—and products thereof—of generation of nucleic acids (RNA) or the generation of a peptide or polypeptide, also referred to as transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The terms allele expression, allele-specific expression, or allelic expression in the context of this specification refers to a measure of gene expression that is attributed to a single copy, or allele at a specific genetic locus. Allelic expression depends on genotype i.e. whether a subject is homozygous, or heterozygous at a specific nucleic acid region or single base pair position, as well as copy number i.e. relative number of copies of each allele, and other forms of regulation that may affect the transcription rate of one, or both copies of the gene. Allelic expression can be defined as a ratio, or relative expression comparing matched alleles, most often a reference and alternative alleles at the same genetic locus in a heterozygous individual. If allelic expression of the matched alleles is imbalanced, i.e. one is expressed more than the other, this is termed allelic expression imbalance, or differential allelic expression.

Allelic expression imbalance, or differential allelic expression, in the context of the present specification refers to two alleles of a genetic locus in a heterozygous individual which are expressed or regulated at different rates. For example, when considering the total amount of mRNA transcripts in a sample characterised by the presence of either reference and alternative allele SNP rs2628315, in the absence of allelic expression imbalance, half of the total STXBP4 mRNA present will be derived from the alternative adenine (A) allele, and half from the reference guanine (G) allele. However, if significantly more than half of mRNA transcripts of this genetic locus are derived from one allele, this is defined as allelic expression imbalance or differential allelic expression.

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine.

### DETAILED DESCRIPTION OF THE INVENTION

Genetic screening methods aim to determine whether a subject has either no more than an average risk of developing a cancer within their lifetime, or an above average risk of developing cancer, compared to the general population. A first aspect of the invention relates to a method of screening a subject for an inherited genetic risk of being diagnosed with a form of malignant cancer. A subject can be a patient, or healthy person, who wishes to know whether they have an increased risk of developing cancer, for example, a subject who has one or more relatives who have developed a type of cancer where genetic predisposition is known to play a role, for example, lung, bladder, breast, prostrate, or ovarian cancer. The term subject further encompasses patients who have been diagnosed with cancer, so as to determine whether their disease may be linked to a genetic predisposition in order to inform family planning, or preventative interventions that may limit disease recurrence. The subject may be a relative of a patient who has been diagnosed with cancer.

The method according to the invention is applied to a sample comprising genetic material obtained from the subject. In some embodiments, the sample is a tissue sample associated with the malignant cancer, for example a tissue biopsy. Such samples are particularly useful being sensitive to both somatic and inherited mutations. In particular embodiments, the sample is a non-invasive sample, for example a blood sample.

The method according to this first aspect of the invention comprises first measuring (quantifying) the relative amount, or level of mRNA transcript of a reference allele and an alternative allele at a risk SNP, or at a plurality of risk SNP sites (see section entitled Risk SNP below). In particular embodiments, one or more of the risk SNP is located in a transcribed region of STXBP4. In other particular embodiments, one or more of the risk SNP is located in a transcribed region of COX11. In still other particular embodiments, one or more of the risk SNP is located in a transcribed region of TOM1L1.

In some embodiments of the method according the invention, the method is applied to a subject who has been characterised as heterozygous for said risk SNP. The method is particularly useful in heterozygous individuals, as subjects who are not characterised by an alternative allele at any of the risk SNP can be excluded from hereditary risk derived from the presence of alternative alleles. The method provides more accuracy at determining risk than the presence of an alternative allele alone. The process of genotyping a subject for the presence of absence of risk SNP is a standard procedure, comprising allele-specific measurements of reference and SNP alleles in mRNA or DNA extracted by standard methods from a tissue sample obtained from the test subject. In some embodiments, the patient is screened for the presence of alternative alleles prior to application of the method, for example using genome sequencing. In other embodiments, the determination of the subject's genotype for each of the one or more risk SNP is made at the same time as the mRNA measurements of reference and wildtype alleles of risk SNP according to the invention, for example, using quantitative real time PCR or mRNA sequencing.

The next step of method according the invention is the assignment of a risk, or probability of developing the malignant cancer. An increased risk of developing cancer is assigned to subjects characterised by significant over-expression of either the alternative allele or the reference allele mRNA in a direction previously determined to be associated with samples obtained from patients diagnosed with cancer.

The level, and direction of the expression (in other words, whether the reference, or alternative allele is expressed at a greater rate) that denotes the presence of increased risk of malignant cancer according to the invention is predetermined by analysis of the relative alternative allele expression of each risk SNP according to the invention in nucleic acid samples obtained from a cohort of subjects for whom the disease status is known, where roughly half are healthy subjects (see Direction and thresholds for over-expression of risk SNP alleles below).

For risk SNP wherein DAE in cases and controls can be described by a log₂(mean alternative allele expression levels/mean reference allele expression level) of a cohort of healthy controls, which is larger than a log₂(mean alternative allele expression levels/mean reference allele expression level) of a cohort of patients diagnosed with the malignant cancer, an above average risk of being diagnosed with the malignant cancer is assigned if:

the reference allele expression level is significantly >than the alternative allele expression level for the risk SNP. This is the case for the risk SNP rs17817901, rs12936860, and rs2628315 studied in the examples.

For risk SNP wherein the inverse direction of DAE is associated with disease, i.e. where a log₂(mean alternative allele expression levels/mean reference allele expression level) of a cohort of healthy controls, is less than a log₂(mean alternative allele expression levels/mean reference allele expression level) of a cohort of patients diagnosed with the malignant cancer, an above average risk of being diagnosed with the malignant cancer is assigned if:

the reference allele expression level is significantly <than the alternative allele expression level the risk SNP.

Conversely, a no more than average probability of developing the malignant cancer is assigned to subjects for whom each of the one or more SNP, the sample is characterised by no significant over-expression of either the alternative allele or the reference allele mRNA, or where one of the alleles is over-expressed, but in a direction not previously determined to be associated with disease risk. A subject may also be assigned a no more than average risk if genotyping indicates they do not express an alternative allele associated with disease risk at any one of the risk SNP, in other words, if they are not characterised by a genotype associated with disease risk.

In some embodiments of the method according to the invention, the method predicts the risk of a subject of developing a cancer associated with the 17q22 risk locus. In particular embodiments, the malignant cancer is ovarian cancer. In alternative embodiments, the malignant cancer is lung cancer. In still further alternative embodiments, the cancer is prostate cancer. In alternative embodiments, the cancer is bladder cancer. In particular embodiments, method determines the subjects risk of receiving a diagnosis of breast cancer.

### Risk SNP

Studies of genetic risk such as genome wide association studies (GWAS) identify gene alternative sequences (A) that differ from the more common reference (R) genetic sequence of the human genome, which are present significantly more often in patients with a heritable disease than subjects who do not have the disease. These alleles, most often single nucleotide polymorphisms (SNP), are classified as “risk” alleles, or “lead-risk SNP”, if the likelihood of this association with disease occurring by chance is less than a genome-wide significance threshold, wherein a parametric, or non-parametric test comparing the how often the allele occurs between cases and controls yields a p value (p)<10⁻⁸.

The term risk SNP refers to SNP of particular utility as biomarkers according to the invention, such as rs17817901, rs12936860, and rs2628315, or others in linkage disequilibrium with these SNP. As the method harnesses the predictive power of multiple risk SNP with differential gene transcription caused by shared regulatory elements, a risk SNP according to the invention encompasses only SNP located within the introns or exons of a transcribed gene. In addition, a risk SNP according to the invention is characterised by a non-zero mean difference between cases and controls, for example when the distribution of the allelic expression ratios of the expression level of the reference and alternative allele are compared in cases and controls using a non-parametric or parametric test, they yield a mean difference with confidence interval excluding zero or significance of p<10⁻², or less.

In some embodiments of the method according to the invention, one or more of the risk SNP are found significantly more frequently in patients with cancer, than healthy controls who have not been diagnosed with cancer. For example, an SNP which has been found to occur more frequently than can be observed by chance in tissue samples, most frequently blood samples, in cases than controls in a GWAS, or other large scale genetic study. A p value of less than 10⁻⁸ is one commonly accepted threshold for a significant association with cancer in such studies.

As a single SNP is rare in the population, in particular embodiments, the assay incorporates information from a plurality of surrounding SNPs in linkage with one or more powerfully determinant SNP that are both common and disruptive enough to be identified in a GWAS. By incorporating information from surrounding SNPs, which may be less common, or disruptive, but which are found in the same locus, more patients can be identified with the same risk of disease, delivering a more powerful tool for genetic counselling purposes. Example 1 demonstrates that risk association between the SNP rs2787486 linked with breast cancer incidence in a GWAS study may also be detected by analysis of differential allelic expression in a case-control association analysis of the risk SNP rs17817901, rs2628315 or rs12936860, SNP which are in strong, or partial linkage disequilibrium with the risk SNP rs2787486.

In particular embodiments of the method according to the invention, an allele expression level is determined for one or more risk SNP in linkage disequilibrium with a risk SNP identified in a GWAS study as specified in the paragraph above. Linkage disequilibrium may be defined, for example, by a Pearson coefficient of correlation (r²) of at least (>) 0.2, in other words, a low level of linkage disequilibrium. In other embodiments, the risk SNP examined in the assay is in at least partial linkage disequilibrium with an SNP correlated with disease in a GWAS study, defined as a r² value >0.6.

In particular embodiments of the method according to the invention, an allele expression level is determined for alleles of one or more risk SNP present in ≥1% the general population, as this will identify patients bearing a risk SNP alternative allele which occur frequently in the general population, such as, rs17817901, rs2628315 or rs12936860.

In particular embodiments, the one or more risk SNP are in linkage disequilibrium with rs2787486, located within the known cancer risk locus 17q22.

In other embodiments of the method according to the invention, AE of the reference and alternative allele for rs3211416 is determined.

In some embodiments, the method according to the invention comprises determining whether DAE of the reference allele of SNP rs17817901 is present in a nucleic acid sample obtained from a patient, and assigning the subject a risk of developing cancer, particularly breast cancer if over-expression of the reference allele is observed.

In some embodiments, the method according to the invention comprises determining whether DAE of the reference allele of SNP rs2628315 is present in a nucleic acid sample obtained from a patient, and assigning the subject a risk of developing cancer, particularly breast cancer if over-expression of the reference allele is observed.

In some embodiments, the method according to the invention comprises determining whether DAE of the reference allele of SNP rs12936860 is present in a nucleic acid sample obtained from a patient, and assigning the subject a risk of developing cancer, particularly breast cancer if over-expression of the reference allele is observed.

In some embodiments, the method according to the invention comprises determining whether DAE of the reference allele of SNP rs12936860 or SNP rs17817901 is present in a nucleic acid sample obtained from a patient, and assigning the subject a risk of developing cancer, particularly breast cancer if over-expression of either reference allele is observed.

In some embodiments, the method according to the invention comprises determining whether DAE of the reference allele of SNP rs2628315 or SNP rs17817901 is present in a nucleic acid sample obtained from a patient, and assigning the subject a risk of developing cancer, particularly breast cancer if over-expression of either reference allele is observed.

In particular embodiments of the method according to the invention, the method comprises
a. determining the genotype for the risk SNP rs17817901 in a sample obtained from the subject, particularly a blood sample, and
b. in a measurement step, if the subject is heterozygous for a reference allele and an alternative allele, determining for said risk SNP rs17817901

an mRNA expression level of a reference allele, and

an mRNA expression level of the alternative allele,
1. to provide a reference allele expression level and an alternative allele expression level; and
b. in a risk assignment step, assigning the subject:

an above average risk of being diagnosed with breast cancer, if the reference allele expression level is significantly more than (>) the alternative allele expression level; or

a no more than an average risk of being diagnosed with breast cancer if
a. the subject is homozygous for the reference allele, or
b. the reference allele expression level is equal to or significantly less than (≤) the alternative allele expression level.

In particular embodiments of the method according to the invention, the method comprises
determining the genotype for the risk SNP rs12936860 in a sample obtained from the subject, particularly a blood sample, and
in a measurement step, if the subject is heterozygous for a reference allele and an alternative allele, determining for said risk SNP rs12936860
an mRNA expression level of a reference allele, and
an mRNA expression level of the alternative allele,
   1. to provide a reference allele expression level and an alternative allele expression level; and
in a risk assignment step, assigning the subject:
an above average risk of being diagnosed with breast cancer, if the reference allele expression level is significantly more than (>) the alternative allele expression level; or
a no more than an average risk of being diagnosed with breast cancer if
a. the subject is homozygous for the reference allele, or
b. the reference allele expression level is equal to or significantly less than (≤) the alternative allele expression level.

In particular embodiments of the method according to the invention, the method comprises
determining the genotype for the risk SNP rs2628315 in a sample obtained from the subject, particularly a blood sample, and
in a measurement step, if the subject is heterozygous for a reference allele and an alternative allele, determining for said risk SNP rs2628315
an mRNA expression level of a reference allele, and
an mRNA expression level of the alternative allele,
   1. to provide a reference allele expression level and an alternative allele expression level; and
in a risk assignment step, assigning the subject:
an above average risk of being diagnosed with breast cancer, if the reference allele expression level is significantly more than (>) the alternative allele expression level; or
a no more than an average risk of being diagnosed with breast cancer if
a. the subject is homozygous for the reference allele, or
b. the reference allele expression level is equal to or significantly less than (≤) the alternative allele expression level.

### Direction and Thresholds for Over Expression of Risk SNP Alleles

In particular embodiments of the method according to the invention, DAE, in other words, significant over expression of a risk SNP reference or alternative allele signifies an expression level above, or below a threshold. The threshold, indicating both an amount, and direction of over expression (i.e. which of the alleles is more strongly expressed) associated with disease, may be obtained by comparing allelic expression (AE) ratios calculated from measurements on mRNA allelic expression levels obtained from a cohort of heterozygous individuals, where about half of the subjects have previously been diagnosed with said hereditary cancer disease, and about half are healthy subjects. A significant difference in AE ratio distribution is characterised by a p value of p<10⁻² using a parametric or non-parametric test to compare the difference in distribution of the reference and alternative allele in cases and controls, or a non-zero difference in estimation statistical methods.

In some embodiments, the threshold, or cut off for significant expression of an allele may be a fold change comparing reference and alternative allele expression levels.

The reference and alternative alleles have varying frequencies among populations and the frequencies provided herein are based on the allele frequency in the European population. The direction and amount of differential allele expression serving as a threshold for risk of developing the disease, should be based on appropriate assessment of the prevalence of DAE in a genetically representative cohorts of patients and healthy controls.

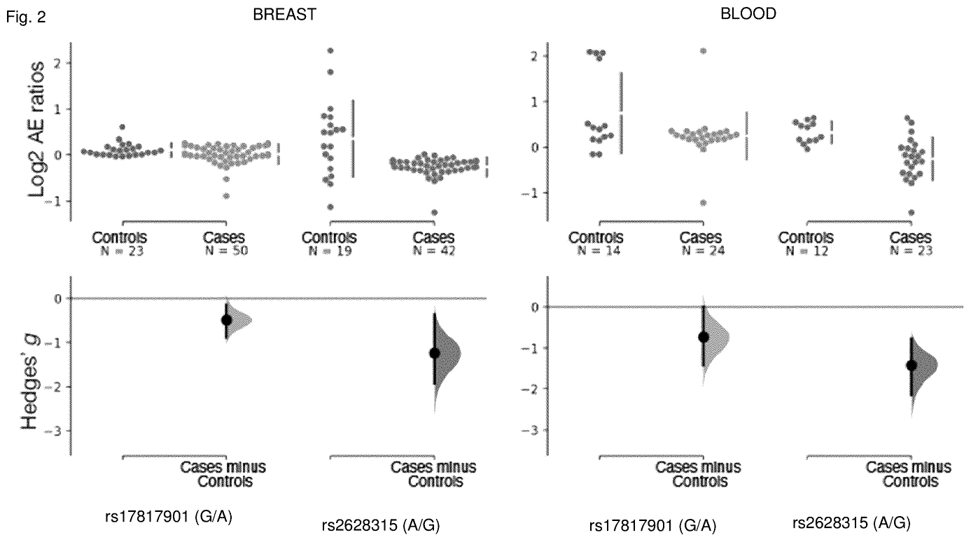

In particular embodiments, the method according to the invention comprises measuring the expression of the risk SNP rs17817901. The subject is assigned an above average risk of developing cancer if the mRNA expression level of a reference rs17817901 adenine (A) allele is ≥about 2-fold the mRNA expression level of an alternative rs1781790 guanine (G) allele. FIG. 2 demonstrates that rs17817901 A allele is expressed on average twice as much in breast cancer patients as in healthy controls in both blood and breast tissue samples.

In particular embodiments, the method according to the invention comprises measuring the expression of the risk SNP rs12936860. The subject is assigned an above average risk of developing cancer if the mRNA expression level of a reference rs12936860 G allele is ≥about 2-fold the mRNA expression level of an alternative rs12936860 A allele. Alternatively, the subject is assigned a no more than average risk of cancer if the expression of the G allele is below this threshold, with the proviso that they do not display DAE at other risk SNP.

An association between AE ratios measured at rs17817901, located in a genomic region shared by TOM1L1 and COX11, was also associated with breast cancer in Example 1. Patient samples more often preferentially expressed the reference A rs17817901 allele, which is frequently linked to the risk-associated A-rs2787486 allele. rs17817901 is in strong LD with the risk lead-variant rs2787486 (r²=0.74) and even stronger LD (r²=0.85) with a previously associated variant rs6504950 (OR=0.95; 95% CI: 0.92-0.97; p-value=1.4×10⁻⁸)⁸. Here too, a shift was observed from the controls preferentially expressing the protective G-rs17817901 allele to the patients preferentially expressing the risk A-rs17817901 allele. On average, patients express the risk-associated A-rs17817901 allele 2-fold more than the controls. FIG. 2 demonstrates that rs17817901 A allele is expressed on average twice as much in breast cancer patients as in healthy controls in both blood and breast tissue samples, and Table 1 demonstrates that rs12936860 is in complete linkage disequilibrium, and can thus be predicted to offer a similar predictive relationship.

In other particular embodiments of the method according to invention, the expression of a reference G allele and an alternative A allele is measured at the risk SNP rs2628315. The subject is assigned an above average risk of developing cancer if the mRNA expression level of a reference rs2628315 G allele is >about 1.5-fold the mRNA expression level of an alternative rs2628315 A allele. The most significant association with breast cancer was for the AE ratios measured at rs2628315, in an intron of STXBP4. This variant is in complete LD with rs2787486, the strongest risk association reported in this locus (OR=0.92; 95% CI: 0.90-0.94; p-value=8.96×10⁻¹⁵) The G rs2628315 allele, proxy to the risk A rs2787486 allele, is 1.5-fold more expressed in cases.

In alternative embodiments, the threshold is applied to a ratio calculated between the two allele expressions in heterozygous cases and controls. In particular embodiments, the cut-off value indicating risk associated DAE of the reference or the alternative allele, is a normalised measure of effect size comparing the DAE of heterozygous patients and controls, such as a Hedges G value.

The data present in example 1 shows that such a threshold can be obtained by comparing allele expression measurements in a cohort of ≥20 subjects, where roughly half have been diagnosed with breast cancer, and the remaining subjects are healthy controls. The examples demonstrate thresholds which differentiate the AE of cancer patients and controls of each of four risk SNP according to the invention. Some methods were validated in blood, others in solid tissue (FIG. 2, Tab. 2). The threshold should be predetermined in samples that match those used in the method according to the invention.

In some embodiments of the method according to the invention, the measurement step is followed by a calculation step, wherein an AE ratio is determined for each of the one or more risk SNPs. To determine the AE ratio, the reference allele expression level is divided alternative allele expression level obtained from the subjects sample, or vice versa. Different thresholds would be applied if the AE ratio is obtained by dividing the alternative allele expression level by the reference allele expression level.

The description of the method in the following paragraph relates to a process of assignment of risk to a subject assuming the AE has been calculated by normalising the ratio of the reference allele expression, relative to the alternative allele expression. This process has also been applied to allelic expression measurements obtained from both cases and controls, to yield an amount, and a direction of the expression which serves as a threshold to be applied for assessment of subject samples in a method according to the invention applied for genetic screening. In certain particular embodiments, this normalised AE ratio is calculated according to the formula:

Log₂(Alternative allele expression level/Reference allele expression level)

In particular embodiments of the method according to the invention, in the risk assignment step, for risk SNP wherein DAE in cases and controls can be described by a log₂(mean alternative allele expression levels/mean reference allele expression level) of a cohort of healthy controls, which is larger than a log₂(mean alternative allele expression levels/mean reference allele expression level) of a cohort of patients diagnosed with the malignant cancer:

an above average risk of being diagnosed with the malignant cancer is assigned if the AE ratio is ≤an AE threshold for the risk SNP, as for the risk SNP rs17817901, rs12936860, and rs2628315 studied in the examples. Conversely, a no more than average risk is assigned for said risk SNP if the AE ratio is >the AE threshold.

For risk SNP wherein the inverse direction of DAE is associated with disease, i.e. where a log₂(mean alternative allele expression levels/mean reference allele expression level) of a cohort of healthy controls, is less than a log₂(mean alternative allele expression levels/mean reference allele expression level) of a cohort of patients diagnosed with the malignant cancer:

an above average risk of being diagnosed with the malignant cancer is assigned if the AE ratio is ≥an AE threshold for the risk SNP. Conversely, a no more than average risk is assigned for said risk SNP if the AE ratio is <the AE threshold.

In particular embodiments of the method according to the invention, the AE threshold for one or more of the risk SNP is a normalised measure of the effect size comparing the difference between:

the mean AE ratio obtained from samples of a cohort of ≥about 10 patients heterozygous for said risk SNP having been diagnosed with said hereditary cancer disease, and

the mean AE ratio obtained from samples from a cohort of ≥about 10 healthy subjects heterozygous for said risk SNP.

In more particular embodiments of the method according the invention, the threshold is normalised measure of effect size. The use of normalized AE ratio distributions confers robustness to rSNP mapping purposes, as it isolates the effect of cis-regulatory variation. In some embodiments, the AE threshold is a value obtained by a Mann Whitney between the groups in the paragraph above. In others, the measures is Cohen's D comparison. Still more particularly, the AE threshold is derived from a Hedge's g test value obtained from a comparison of healthy samples and samples derived from cancer patients.

In certain embodiments of the method according to the invention where the sample is a blood sample, and the AE ration is the log 2(Alt/Ref), the AE threshold for rs17817901 or rs12936860 is within the range of −1.6 to 0.18. In particular embodiments, the threshold below which a risk is assigned to a subject is about 0.-7. These risk SNP predictive of malignant breast cancer are in complete linkage disequilibrium, and thus the threshold calculated in the examples for rs17817901 must apply equally to rs12936860, as they are inherited together. In other particular embodiments, where one or more of the risk SNP is rs2628315, and the sample is a blood sample, the AE threshold is within the range of −2.3 to −0.7. in more particular embodiments the threshold is about −1.4.

In alternative embodiments of the method according to the invention relating to prediction of a risk of developing malignant breast cancer the sample comprises, or essentially consists of breast tissue. In more particular embodiments, the sample is breast tissue, and the AE ratio is the log 2(Alt/Ref), and the AE threshold applied to rs17817901 and/or rs12936860 is within the range of −1 to −0.1. In still more particular embodiments, the threshold for either of these risk SNP is about −0.5. In other particular embodiments, where one or more of the risk SNP is rs2628315, and the sample is a breast tissue sample, the AE threshold is within the range of −2 to −0.27. In more particular such embodiments, the threshold is about −1.2.

### Methods of Measuring AE of Risk SNP

In particular embodiments of the method according to the invention, a blood sample is used to determine the mRNA expression level of risk SNP. In alternative embodiments, a tissue sample obtained from an organ associated with the cancer in question is used, for example biopsy sample from a therapeutic or diagnostic surgical intervention. In particular embodiments where the method according to the invention is used to predict a risk of developing malignant breast cancer, the sample is breast tissue.

The method by which the expression levels of risk SNP alleles are measured at the mRNA level is not limited according to the invention, and includes the use of allele-specific nucleic acid probes (reverse complement of the alternative allele and the reference allele for each risk SNP), particularly with a quantitative PCR methodology such as real time PCR, or qPCR, sequencing reactions, or a nucleic acid array. In other embodiments, the risk SNP expression status is determined by mRNA sequencing.

One methodology that is particularly useful for measuring the allelic expression level of risk SNP according to the invention, is a nucleic acid amplification method conducted using polymerase chain reaction of the RNA extracted from the patient tumour sample. A cycle threshold is an example of a quantitative nucleic acid measurement, for example a measurement made with a quantitative polymerase chain reactions (qPCR). This method involves repeated cycles of nucleic acid amplification using nucleic acid probes which hybridise a target wildtype and mutant allele, to generate a product emitting a fluorescent signal, which can be measured to determine the amount of starting genetic material. The cycle threshold may be an average value, or the average value of a number of replicate samples. Other quantitative measurements may substitute the cycle threshold, such as a crossing point, or an adjusted inflexion point.

The skilled artisan will appreciate in embodiments wherein a risk SNP allele expression level is measured by qPCR, values may be expressed examples of differential cycle thresholds compared to a house keeping gene, i.e. the number of qPCR cycles needed to generate a fluorescence signal from the specific nucleic acid probes used, above a user-defined threshold. Optionally, expression levels may be expressed as a value reflecting the different between measurements of the risk SNP, and a control gene, sometimes termed a delta CT. In some embodiments, the expression level is a quantitative measure determined with reference to a standard curve. Expression levels reflect the PCR conditions and cycle threshold, and the exact values of a threshold for pathogenic expression may expected to vary from those derived in the examples from mRNA sequencing samples. Similar thresholds relevant for cancer outcomes for qPCR measurements of allelic expression can be generated by assessing a cohort of similar samples using the same methodology, and performing a correlation analysis with patient outcomes. Specific nucleic acid probes can identify expression of a risk SNP alternative and reference allele with a primer comprising the complementary sequence, using for example, standard TAQman ABI or Sybrgreen enzyme qPCR assay conditions.

The method may be embodied by way of a computer-implemented method, particularly wherein the evaluation and the assignment step are executed by a computer. Further, the method may be embodied by way of a computer program, comprising computer program code, that when executed on the computer cause the computer to execute at least the evaluation and/or assignment step. Particularly, the results of the measurement step may be provided to the computer and/or the computer program by way of a user input and/or by providing a computer-readable file comprising information regarding the expression level obtained during the measurement step. Results from the measurement step may be stored for further processing on a memory of the computer, on a non-transitory storage medium.

Wherever alternatives for single separable features such as, for example, risk SNP, thresholds, or medical indications are laid out herein as “embodiments”, it is to be understood that such alternatives may be combined freely to form discrete embodiments of the invention disclosed herein. Thus, any of the alternative embodiments for a risk SNP may be combined with any of the alternative embodiments of a threshold and these combinations may be combined with to diagnose risk for a medical indication mentioned herein.

The invention further encompasses the following numbered items.

A method to quantify a subject's genetic risk of being diagnosed with a malignant cancer selected from lung, bladder, breast, prostrate, or ovarian cancer, said method comprising:
a. determining the genotype for each of one or more risk SNP in a sample obtained from the subject, particularly a blood sample,
   i. wherein the one or more risk SNP are selected from the list consisting of selected from:
   ii. rs17817901, rs12936860, and/or rs2628315; or
   iii. a SNP in linkage disequilibrium with a risk SNP selected from rs17817901, rs12936860, and/or rs2628315,
      1. wherein linkage disequilibrium is defined by a Pearson coefficient of correlation (r²) of at least (>) 0.2, more particularly an r² value >0.6;
      2. and
b. in a measurement step, if the subject is heterozygous for a reference allele and an alternative allele, determining for one or more risk single nucleotide polymorphisms (SNP),

an mRNA expression level of a reference allele, and

an mRNA expression level of the alternative allele,
a. to provide a reference allele expression level and an alternative allele expression level; and in a risk assignment step, assigning the subject:

an above average risk of being diagnosed with the malignant cancer, if for at least one of one or more risk SNP:
a. the reference allele expression level is significantly more than (>) the alternative allele expression level,
   a. wherein for the risk SNP the log₂(mean alternative allele expression levels/mean reference allele expression level) of a cohort of healthy controls, is >log₂(mean alternative allele expression levels/mean reference allele expression level) of a cohort of patients diagnosed with the malignant cancer, or
b. the alternative allele expression level is significantly >the reference allele expression level,
   a. wherein for the risk SNP the log₂(mean alternative allele expression levels/mean reference allele expression level) of a cohort of healthy controls, is <log₂(mean alternative allele expression levels/mean reference allele expression level) of a cohort of patients diagnosed with the malignant cancer, or

a no more than an average risk of being diagnosed with the malignant cancer if for each of the one or more risk SNP:
a. the subject is homozygous for the reference allele, or
b. the reference allele expression level is equal to or significantly less than (≤) the alternative allele expression level,
   1. wherein for the risk SNP the log₂(mean alternative allele expression levels/mean reference allele expression level) of a cohort of healthy controls, is >log₂(mean alternative allele expression levels/mean reference allele expression level) of a cohort of patients diagnosed with the malignant cancer, or
c. the alternative allele expression level is ≤reference allele expression level,
   1. wherein for the risk SNP the log₂(mean alternative allele expression levels/mean reference allele expression level) of a cohort of healthy controls, is <log₂(mean alternative allele expression levels/mean reference allele expression level) of a cohort of patients diagnosed with the malignant cancer.

The method according to item 1, wherein each of the one or more risk SNP is located within a transcribed region of a gene selected from STXBP4, COX11 and/or TOM1L1.

The method according to item 1 or 2, wherein each of the one or more risk SNP is present in the general population at a frequency of at least (≥) 1%.

The method according to any one of the items 1 to 3, wherein the one or more risk SNP comprises, or is rs17817901;
i. wherein the subject is assigned an above average risk of developing said malignant cancer if the mRNA expression level of a reference rs17817901 adenine (A) allele is ≥about 2-fold the mRNA expression level of an alternative rs1781790 guanine (G) allele.

The method according to any one of the items 1 to 4, wherein the one or more risk SNP comprises or is rs12936860;
i. wherein the subject is assigned an above average risk of developing said malignant cancer if the mRNA expression level of a reference rs12936860 G allele is ≥about 2-fold the mRNA expression level of an alternative of rs12936860 A allele.

The method according to any one of the items 1 to 5, wherein the one or more risk SNP comprises or is rs2628315;
i. wherein the subject is assigned an above average risk of developing said malignant cancer if the mRNA expression level of a reference rs2628315 G allele is ≥about 1.5-fold the mRNA expression level of an alternative rs2628315 A allele.

The method according to any one of the items 1 or 6, wherein in the measurement step, the mRNA expression level is determined for:

rs17817901, and/or rs12936860, and

rs2628315.

The method according to any one of the items 1 to 7, wherein the sample is a blood sample.

The method according to any one of the items 1 to 7, wherein the sample comprises, or essentially consists of breast tissue.

The method according to any one of the items 1 to 9, wherein the measurement step is followed by:
a calculation step, wherein an allelic expression (AE) ratio is determined for each of the one or more risk SNPs,
   1. wherein the AE ratio is obtained using the formula:
b. log₂(alternative allele expression level/reference allele expression level); and
c. wherein in said risk assignment step, the subject is assigned:
an above average risk of being diagnosed with the malignant cancer, if for at least one of one or more risk SNP:
a. the AE ratio is ≤an AE threshold,
   a. wherein the mean AE ratio as specified in a. of a cohort of healthy controls is >the mean AE ratio of a cohort of patients diagnosed with the malignant cancer, or
b. the AE ratio is ≥the AE threshold,
   a. wherein the mean AE ratio of a cohort of healthy controls is <the mean AE ratio of a cohort of patients diagnosed with the malignant cancer;
   ii. or
a no more than average risk of being diagnosed with the malignant cancer if for all of the one or more risk SNP:
the AE ratio is >the AE threshold,
   1. wherein the mean AE ratio of a cohort of healthy controls is >the mean AE ratio of a cohort of patients diagnosed with the malignant cancer, or
the AE ratio is <the AE threshold,
   1. wherein the mean AE ratio of a cohort of healthy controls is <the mean AE ratio of a cohort of patients diagnosed with the malignant cancer.

The method according to item 10, wherein the AE threshold for the one or more risk SNP is a normalised measure of the effect size comparing the difference between:
the mean AE ratio obtained from samples of a cohort of ≥about 10 patients characterised as heterozygous for said risk SNP, and each patient having been diagnosed with said malignant cancer, and
the mean AE ratio obtained from samples from a cohort of ≥about 10 healthy subjects heterozygous for said risk SNP;
   i. particularly wherein the normalised measure of effect size is a measure selected from a Mann Whitney, Cohen's D, or Hedge's g test.

The method according to items 10 or 11, wherein the sample is a blood sample, and wherein the AE threshold for:

rs17817901 or rs12936860 is within the range of −1.6 to 0.18, particularly wherein the threshold is about −0.7, and/or

rs2628315 is within the range of −2.3 to −0.7, particularly wherein the threshold is about −1.4.

The method according to items 10 or 11, wherein the malignant cancer is breast cancer and the sample comprises, or essentially consists of breast tissue;
i. wherein the AE threshold is

rs17817901 or rs12936860 is within the range of −1 to −0.1, particularly wherein the threshold is about −0.5, and/or

rs2628315 is within the range of −2 to −0.2, particularly wherein the threshold is about −1.2.

The method according to any one of the items 1 to 13, wherein the mRNA expression levels are obtained by allele-specific, quantitative, mRNA measurement methodology, particularly a methodology selected from mRNA sequencing, microarray, or real-time quantitative polymerase chain reaction.

The method according to any one of the items 1 to 14, wherein the malignant cancer is breast cancer.

The invention further encompasses the following lettered items.

A method to quantify a subject's genetic risk of being diagnosed with a malignant cancer selected from lung, bladder, breast, prostrate, or ovarian cancer, said method comprising:
a. determining the genotype for each of one or more risk SNP in a sample obtained from the subject, particularly a blood sample,
   i. and particularly wherein one or more of the risk SNP is selected from a SNP located in a transcribed region of a gene selected from the list consisting of STXBP4, COX11 and/or TOM1L1, more particularly a SNP selected from rs17817901, rs12936860, and/or rs2628315; and
b. in a measurement step, if the subject is heterozygous for a reference allele and an alternative allele, determining for one or more risk single nucleotide polymorphisms (SNP),
   i. an mRNA expression level of a reference allele, and
   ii. an mRNA expression level of the alternative allele,
c. to provide a reference allele expression level and an alternative allele expression level; and in a risk assignment step, assigning the subject:
   i. an above average risk of being diagnosed with the malignant cancer, if for at least one of one or more risk SNP:

the reference allele expression level is significantly more than (>) the alternative allele expression level,
a. wherein for the risk SNP the log₂(mean alternative allele expression levels/mean reference allele expression level) of a cohort of healthy controls, is >log₂(mean alternative allele expression levels/mean reference allele expression level) of a cohort of patients diagnosed with the malignant cancer, or

the alternative allele expression level is significantly >the reference allele expression level,
a. wherein for the risk SNP the log₂(mean alternative allele expression levels/mean reference allele expression level) of a cohort of healthy controls, is <log₂(mean alternative allele expression levels/mean reference allele expression level) of a cohort of patients diagnosed with the malignant cancer,
b. or
ii. a no more than an average risk of being diagnosed with the malignant cancer if for each of the one or more risk SNP:

the subject is homozygous for the reference allele, or

the reference allele expression level is equal to or significantly less than (≤) the alternative allele expression level,
a. wherein for the risk SNP the log₂(mean alternative allele expression levels/mean reference allele expression level) of a cohort of healthy controls, is >log₂(mean alternative allele expression levels/mean reference allele expression level) of a cohort of patients diagnosed with the malignant cancer, or

the alternative allele expression level is ≤reference allele expression level,
a. wherein for the risk SNP the log₂(mean alternative allele expression levels/mean reference allele expression level) of a cohort of healthy controls, is <log₂(mean alternative allele expression levels/mean reference allele expression level) of a cohort of patients diagnosed with the malignant cancer.

The method according to item A, wherein each of the one or more risk SNP is located within a transcribed region of at least one gene;
i. and wherein the mean reference allele expression level of samples from a cohort of ≥about 10 patients having been diagnosed with said malignant cancer differs significantly from the mean alternative allele expression level of samples from a cohort of ≥about 10 healthy subjects;
   1. and wherein each of the one or more risk SNP is either:

more frequently present in subjects having been diagnosed with said malignant cancer; and/or

in strong linkage disequilibrium with a risk SNP as specified in a.

The method according item A or B, wherein each of the one or more risk SNP is present in the general population at a frequency of at least (≥) 1%;

The method according to any one of the items A to C, wherein the one or more risk SNP comprises, or is rs17817901;
i. wherein the subject is assigned an above average risk of developing said malignant cancer if the mRNA expression level of a reference rs17817901 adenine (A) allele is about 2-fold the mRNA expression level of an alternative rs1781790 guanine (G) allele.

The method according to any one of the items A to D, wherein the one or more risk SNP comprises or is rs12936860;
i. wherein the subject is assigned an above average risk of developing said malignant cancer if the mRNA expression level of a reference rs12936860 G allele is ≥about 2-fold the mRNA expression level of an alternative of rs12936860 A allele.

The method according to any one of the items A to E, wherein the one or more risk SNP comprises or is rs2628315;
i. wherein the subject is assigned an above average risk of developing said malignant cancer if the mRNA expression level of a reference rs2628315 G allele is ≥about 1.5-fold the mRNA expression level of an alternative rs2628315 A allele.

The method according to any one of the items A to F, wherein in the measurement step, the mRNA expression level is determined for:

rs17817901, and/or rs12936860, and

rs2628315.

The method according to any one of the items A to G, wherein the sample is a blood sample.

The method according to any one of the items A to G, wherein the sample comprises, or essentially consists of breast tissue.

The method according to any one of the items A to I, wherein the measurement step is followed by:
a. a calculation step, wherein an allelic expression (AE) ratio is determined for each of the one or more risk SNPs,
   i. wherein the AE ratio is obtained using the formula:
   ii. log 2(alternative allele expression level/reference allele expression level); and
   iii. wherein in said risk assignment step, the subject is assigned:
   iv. an above average risk of being diagnosed with the malignant cancer, if for at least one of one or more risk SNP:

the AE ratio is ≤an AE threshold,
i. wherein the mean AE ratio as specified in a. of a cohort of healthy controls is >the mean AE ratio of a cohort of patients diagnosed with the malignant cancer, or

the AE ratio is ≥the AE threshold,
i. wherein the mean AE ratio of a cohort of healthy controls is <the mean AE ratio of a cohort of patients diagnosed with the malignant cancer;
2. or
ii. a no more than average risk of being diagnosed with the malignant cancer if for all of the one or more risk SNP:

the AE ratio is >the AE threshold,
1. wherein the mean AE ratio of a cohort of healthy controls is >the mean AE ratio of a cohort of patients diagnosed with the malignant cancer, or

the AE ratio is <the AE threshold,
1. wherein the mean AE ratio of a cohort of healthy controls is <the mean AE ratio of a cohort of patients diagnosed with the malignant cancer.

The method according to item J, wherein the AE threshold for the one or more risk SNP is a normalised measure of the effect size comparing the difference between:
the mean AE ratio obtained from samples of a cohort of ≥about 10 patients characterised as heterozygous for said risk SNP, and each patient having been diagnosed with said malignant cancer, and
the mean AE ratio obtained from samples from a cohort of ≥about 10 healthy subjects heterozygous for said risk SNP;
   1. particularly wherein the normalised measure of effect size is a measure selected from a Mann Whitney, Cohen's D, or Hedge's g test.

The method according to item J or K, wherein the sample is a blood sample, and wherein the AE threshold for:

rs17817901 or rs12936860 is within the range of −1.6 to 0.18, particularly wherein the threshold is about −0.7, and/or

rs2628315 is within the range of −2.3 to −0.7, particularly wherein the threshold is about −1.4.

The method according to item J or K, wherein the malignant cancer is breast cancer and the sample comprises, or essentially consists of breast tissue;
a. wherein the AE threshold is

rs17817901 or rs12936860 is within the range of −1 to −0.1, particularly wherein the threshold is about −0.5, and/or

rs2628315 is within the range of −2 to −0.2, particularly wherein the threshold is about −1.2.

The method according to any one of the items A to M, wherein the mRNA expression levels are obtained by allele-specific, quantitative, mRNA measurement methodology, particularly a methodology selected from mRNA sequencing, microarray, or real-time quantitative polymerase chain reaction.

The method according to any one of the items A to N, wherein the malignant cancer is breast cancer.

The invention is further illustrated by the following examples and figures, from which further embodiments and advantages can be drawn. These examples are meant to illustrate the invention but not to limit its scope.

### EXAMPLES

### Material & Methods

### Samples

All samples were collected for this study following written informed consent from all donors. All procedures followed were per the established rules of the Addenbrooke's Hospital Local Research Ethics Committee (REC references 06/Q0108/221, 07/H0308/161, and 04/Q0108/21 for normal breast tissue from healthy controls, normal-matched tissue, and blood from breast cancer patients, respectively) and the Eastern Multicenter Research Ethics Committee (SEARCH Study, Azzato E M., 2010 Brit. J. Canc. 102:1294) The original studies provide the full description of the samples¹³⁻¹⁵.

### Nucleic Acid Preparation and Processing

DNA and total RNA was extracted from rom all samples. cDNA was synthesized using the SuperScript™ First-Strand Synthesis System (Invitrogen), from 50 ng of total RNA and a mixture of oligo(dT)₂₀ and random hexamers, according to the manufacturer's instructions. Target-specific preamplification of cDNA was performed with TaqMan™ PreAmp Master Mix (2×) (Applied Biosystems), pooled TaqMan™ SNP Genotyping Assays (0.2×) (Applied Biosystems), and 1.25 ul of cDNA. Thermal cycling conditions consisted of enzyme activation at 95° C. for 10 min, followed by 8 or 14 cycles of denaturation at 95° C. for 15 sec and annealing/extension at 60° C. for 4 min. Finally, products were diluted 1:5 before use in subsequent reactions.

### Genotyping

Genotyping was performed using TaqMan™ SNP Genotyping Assays (a custom assay for rs17817901 and predesigned assays C_15903698_10 and C_30379485_10, for rs2628315 and rs9899602, respectively), under cycling conditions per the manufacturer's instructions. Reactions were prepared in a final volume of 5 ul with TaqMan™ Universal Master Mix II, with UNG (2×) (Applied Biosystems), TaqMan™ SNP Genotyping Assay (40×) (Applied Biosystems), DNase/RNase-free water, and 8 ng of DNA. Reactions were performed in a Bio-Rad CFX384 system (Bio-Rad).

### Allelic Expression (AE) Analysis

Using previously generated data from a genome-wide microarray study (Liu R, 2020, Bioinformatics 28:1102) allelic expression was quantified (Jacinta-Fernandes, 2019 Npi Genom Med. 5:4). Briefly, the transcribed SNPs (aeSNPs) were identified in the risk locus included in the data, and allelic expression levels extracted for all heterozygous individuals at each aeSNP. We calculated the normalized allelic expression ratios (AE ratios_norm) as the log 2[(expression of alternative allele)/(expression of reference allele)] normalized by the same ratio calculated from genomic DNA data (gDNA) to account for copy number variation and correct for technical biases. To test if the distribution mean of the AE ratios_norm for each aeSNP was equal to zero (null hypothesis), a two-sample Student's t-test was used. An adjusted the p-value significance level for multiple testing used the p.adjust function with the Bonferroni method (stats R package) and defined as differentially allelic expressed SNPs (daeSNPs) those aeSNPs with p.adjust≤0.05.

Allele-specific expression was also quantified using real-time PCR for the case-control association study, with the TaqMan SNP Genotyping Assays indicated above and cDNA, and as described previously (Maia A-T., 2009 Breast Cancer 11:R88). Experiments were performed on 96.96 Dynamic Arrays IFC in the Biomark™ HD system (Fluidigm) and on a CFX384 real-time PCR machine (BioRad). Here, allelic expression ratios were calculated as the log 2[(alternative allele)/(reference allele)], without normalization. Standard curves consisting of serial dilutions of DNA from CEPH lymphoblastoid cell lines heterozygous for each SNP were used to determine the quantitative performance. Cases, controls, standard curves, and at least two no-template controls (NTC) were analyzed in triplicates simultaneously in each experiment, and cases and controls were solely compared within each experiment.

### Case-Control Association Analysis

To detect allelic expression ratios associated with a risk of breast cancer, the inventors calculated the effect size, given with magnitude and direction of the difference, between the AE ratios of cases and controls, in breast tissue and blood. For this purpose, the inventors used the Hedges' g (effect size) test, which is a standardized mean difference method that normalizes for sample size, particularly suitable for small ones (<20 samples in each group). Hedges' g (effect size) test is a standardized mean difference method that normalizes for sample size and is more suitable for small samples sets (<20 samples in each group) compared to other methods. More specifically, each test included taking 5000 bootstrap samples; the confidence interval is bias-corrected and accelerated. Three levels of confidence intervals are presented: 90%, 95% and 98%. The inventors also report the p-value(s) for the likelihood(s) of observing the effect size(s) if the null hypothesis of zero difference is true, assessed by the Two-side Student's t-test. P-values were also permuted with 5000 label reshuffles of the controls and cases. All Cumming estimation plots and statistical tests were performed using the online tool available at www.estimationstats.com (Ho, J. 2019 Nat. Methods, 16:565).

### Example 1

Identifying the target genes regulated by risk-variants usually includes physical interaction studies (e.g., chromatin conformation capture), in-vitro assays evaluating protein binding modification at regulatory elements (e.g., band shifts and transfection assays), and more integrative approaches, but often lacks direct validation of the regulation in-vivo in the complex human genomic context. However, as these variants regulate gene expression in an allele-specific manner, their target genes can be detected by measuring allelic expression levels in heterozygous individuals for a transcribed variant. As gene expression regulation is partly tissue-specific, allelic expression quantification is also more informative when carried in the disease's closest cell type of origin. Hence, it is possible functional characterization of GWAS loci could be improved by: (1) starting with the identification of the target genes under the control of cis-regulatory variants in any given locus via differential allele-specific expression (DAE) studies; (2) followed by the mapping of the causal variants associated with DAE; and (3) finishing with the identification of significant differences in gene expression regulation between patients and healthy individuals.

This approach was tested in the locus 17q22, which was associated with a risk of breast cancer in three studies, including the risk of male breast cancer, and possibly with breast cancer survival. All three genes in the locus have been suggested as targets for the alleles that modify disease risk, but no study has linked them directly to risk. Here, the inventors first analyzed allelic expression patterns in control normal breast tissue to assess whether any or all genes in the locus were under the control of cis-regulatory variants; then, to assess association with risk and identify target genes, compared the distribution of allelic expression ratios measured in the normal breast tissue of patients with that of controls.

Genetic Variants Regulate Genes in 17q22 Risk-Locus in Strong Linkage Disequilibrium with the Lead Risk-SNP

Firstly, COX11, TOM1L1, or STXBP4 were assessed for control by cis-regulatory variants in normal breast tissue. Normalized allelic expression (AE) ratios were calculated at 22 aeSNPs located in the three genes (Table 1). Thirteen (59%) aeSNPs showed significant deviations from equimolar allelic expression and were designated differentially allelic expressed SNPs−daeSNPs (Table 1). The observed differences between alleles reached a maximum of 2.15-fold at rs7643. This identified daeSNPs in all three genes in the locus, suggesting they all are targets of cis-regulatory variation.

The patterns of the AE ratio distributions are indicative of the linkage disequilibrium (LD) between the regulatory variant and the transcribed variant where AE is measured (Xiao R., 2011, Genet. Epidemiol 35: 515). Hence, to test if there is a link between cis-regulation and risk, pairwise LD was tested between the daeSNPs and the locus lead risk-SNP rs2787486, and was then matched to AE ratio distribution patterns. Four daeSNPs showed marked preferential expression of the same allele in all heterozygous individuals tested and were in high LD (r²≥0.6) with rs2787486, suggesting that the same variant could confer risk and regulate gene expression levels.

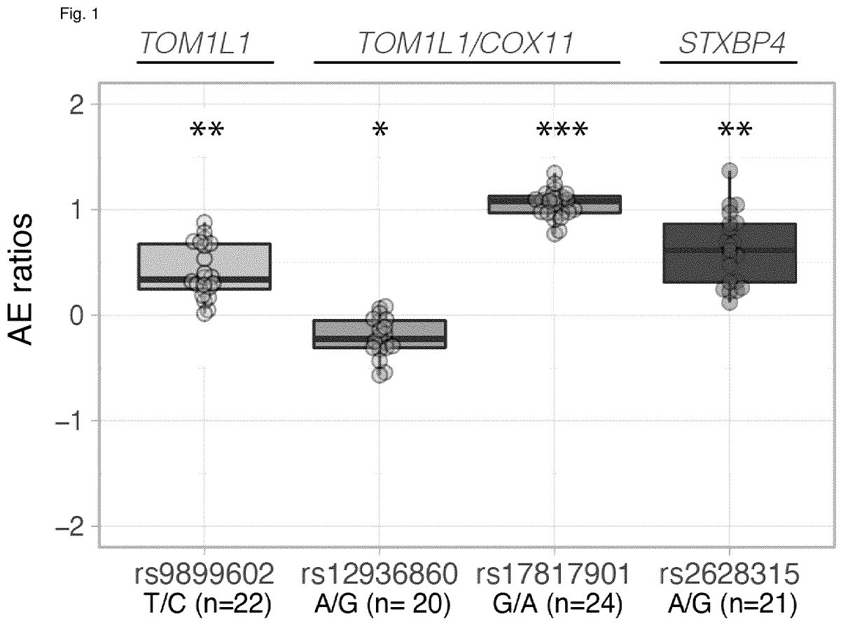

One of these four daeSNPs, rs2628315, is in complete LD with the risk-variant and maps exclusively to the STXBP4 gene (Table 1). At this variant, the allele preferentially expressed is associated with protection against breast cancer (FIG. 1), suggesting that a higher predominance of the A allele is beneficial. Concordantly, the GTEx project reports rs2628315 as an eQTL (expression quantitative trait locus) for STXBP4 expression in mammary tissue (p=9.68E-07).

Another two daeSNPs, rs12936860 and rs17817901, map to a region shared by the TOM1L1 and COX11 genes and are in strong LD with rs2787486 (r²=0.74 for both, Table 1). Of these two daeSNPs, rs17817901 showed the most significant differential allelic expression pattern, in which all heterozygotes preferentially expressed the alternative G allele. This AE ratio distribution is consistent with the daeSNP being in complete LD with the cis-regulatory variant (rSNP) creating the allelic effect which facilitates the mapping of the latter (FIG. 1). Additionally, preferential expression of the alternative G allele could correlate with the protective effect of the alternative C allele of rs2787486.

The fourth daeSNP in high LD with risk-variant rs2787486 is rs9899602 (r²=0.66) that maps exclusively to TOM1L1. It showed preferential expression of the reference T allele, which is correlated to the risk-associated A allele of rs2787486 (FIG. 1).

These results suggest that the differential allelic expression detected in all three genes could be associated with the risk of breast cancer and that all genes are candidate targets for the risk detected in the locus.

AE Ratios in Normal Breast Tissue and Blood Associate with Breast Cancer Risk

Next, to discern between chance colocalization and a true association between allelic expression ratios and risk association, the inventors tested whether risk-causing variants are cis-regulating genes in the 17q22 locus, as the allelic expression ratios they generate should have distinct distributions in patients (cases) and healthy individuals (controls). A case-control association analysis using AE ratios measured in the normal breast as a quantitative phenotype. This analysis was performed for the three daeSNPs displaying the highest LD with the risk-associated variant, each localized in one of the genes in the locus. As rs12936860 and rs17817901 are in complete LD, only rs17817901 was analyzed.

The daeSNP rs2628315, located in an intron of STXBP4, showed the largest effect size (g=−1.237) (Table 2, FIG. 2), and the most significantly different AE ratio distributions. This result shows that the AE ratio distribution in normal breast of cases is shifted towards the preferential expression of the reference G allele, the least expressed in controls. As rs2628315 and the risk-variant rs2787486 are in complete LD, this result suggests that increased risk is associated with the preferential expression of the reference allele of both variants.

The analysis of rs17817901 also revealed a shift in the distribution of AE ratios in cases towards the preferential expression of the reference A allele with an estimated effect size of g=−0.486 (Table 2, FIG. 2). As rs17817901 is in strong LD with the risk-variant rs2787486, our results suggest that risk could be associated with a higher expression of the reference A allele of rs17817901. However, as rs17817901 locates in a genomic region shared by the TOM1L1 and COX11 genes, the inventors considered both genes as candidate target genes for breast cancer.

However, the analysis of the daeSNP rs9899602 did not reveal any significant difference between the two populations, suggesting that TOM1L1 might not be a target gene for the risk detected via the lead-SNP rs2787486 (Table 2, Figure S2). The SNP rs9899602 is the daeSNP in weaker LD with the lead risk-SNP amongst the ones with significant DAE (Table 1). Integrated with the results obtained for rs17817901, this suggests that COX11 is the most likely candidate of the two overlapping genes.

Associations were next examined in blood samples from cases and controls. For the daeSNP rs2628315, a comparable effect size (g=−1.419) and a significant difference was observed in the AE ratio distributions of the two groups, with a concordant shift direction with that observed in breast tissue: preferential expression of the risk-associated G allele of rs2628315.

For the daeSNPs rs17817901, a larger effect size was observed than in breast tissue (g=−0.737), and in concordant direction—cases preferentially expressed the A-rs17817901 allele which is in strong LD with the risk-associated A-rs2787486 allele.

Genetic Variants Regulate Genes in CDC16 Risk-Locus in Strong Linkage Disequilibrium with the Lead Risk-SNP

### Differential Allelic Expression Analysis

DNA and total RNA from 64 samples of normal breast tissue, were analysed using Illumina Exon510S-Duo arrays (humanexon510s-duo), as described (Liu R., Bioinformatics 2012, 28:1102). After normalization, SNPs with average log 2 RNA intensity values lower than 9.5 and less than 5 heterozygous values were excluded from the analysis. A two samples Student's t-test was applied to compare RNA log ratios between heterozygous (AB) and homozygous groups (AA and BB). Only SNPs with p-values lower than 0.05 for all comparisons were further analysed. The following equation was used for normalisation of DAE: log₂((RNA allele A/RNA allele B)/(DNA allele A/DNA allele B)). This analysis was carried out using R and Bioconductor packages as described (Huber W., Nature Pub. Group 2015, 12:115). DAE was inferred when |AE ratio|≥0.58 (1.5 fold or greater difference). This threshold was established based on the sensitivity and specificity of the applied DAE detection method. Linkage disequilibrium (LD) between daeSNPs was evaluated using the genetic variant-centred annotation browser SNiPA (Li Y., Genet Epidemiol. 2010, 34:816).

### Genotype Imputation Analysis on Normal Breast Tissue Samples

Genotype imputation was run on the Illumina Exon 510 Duo germline genotype data from the 64 samples that passed microarrays quality control filters. Prior to imputation data, a quality control was applied to the genotyping data and SNPs with call rates <85%, minor allele frequency <0.01, and Hardy-Weinberg equilibrium with p-value <1.0e⁻⁰⁵ were excluded from the analysis. Genotype data from the chromosome 3 Illumina SNPs that passed quality control was used to impute genotypes at all additional known SNPs in the chromosome using MACH1.0 (Li, Y. et al., Genet. Epidemiol. 34, 816-834, 2010) and the phased haplotypes for HapMap3 release (HapMap3 NCBI Build 36, CEU panel—Utah residents with Northern and Western European ancestry) ©AS reference panel. For imputation with MaCH1.0, a two-step imputation process was used: model parameters (crossover and error rates) were estimated prior to imputation using all haplotypes from the study subjects and running 100 iterations of the Hidden Markov Model (HMM) with the command options: -greedy and -r 100. Genotype imputation was then carried out using the model parameter estimates from the previous round with command options of -greedy, -mle, and -mldetails specified. Imputation results were assessed by the platform-specific measures of imputation uncertainty for each SNP (rsq Score). MaCH -rsq score equals the ratio of the empirically observed variance of the allele dosage to the expected binomial variance p(1-P) at Hardy-Weinberg equilibrium, where p is the observed allele frequency derived from HapMap or estimated from own data. Its value tends to zero if the uncertainty of the imputation results increases. Imputation results were assessed by the platform-specific measures of imputation uncertainty for each SNP (rq Score) and the results were filtered for an rq Score higher than 0.3 (Li Y., Genet Epidemiol. 34:816-834, 2010).

### Differential Allelic Expression (DAE) Mapping Analysis on Normal Breast Tissue Samples

Differential allelic expression mapping analysis was performed by stratifying AE ratios at each CDC16 daeSNP according to the genotype at the genotyped/imputed SNPs located within ±250 Kb of the daeSNP. A two-sample t-test was applied to assess differences between the mean AE ratio between the heterozygous group samples and the combined homozygous groups. P-values were corrected for multiple testing using permutation procedure for N=1000. Permutation-corrected p-values were considered significant below 0.05 and when on average the heterozygous samples displayed larger fold differences between alleles when compared to homozygous samples.

### Results for CDC16 Locus

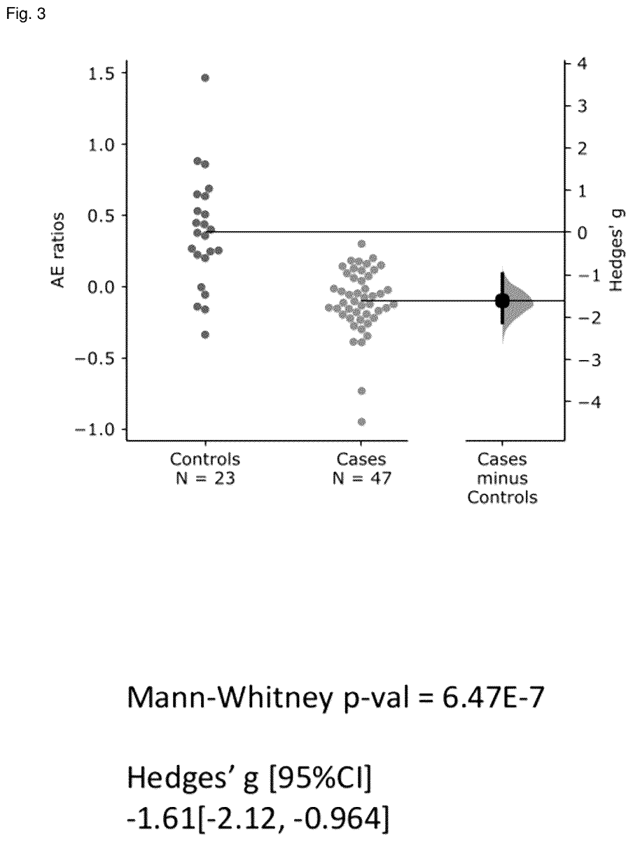

The inventors confirmed the approach for an additional risk locus CDC16. mRNA sequencing was performed on cases and controls for samples of normal breast tissue using an Illumina workflow. A Cumming estimation plot shows the Hedges' g forming a threshold which distinguishes between breast cancer cases and controls for allelic expression ratios was calculated at rs3211416 (ratio calculated as allele A by allele G) in normal breast tissue (FIG. 3).

### Summary

This study finds that in heterozygous individuals, the expression dynamic of the risk allele within a disease risk locus compared to their reference alleles predicts risk of developing cancer much more accurately than genotype, or gene dose. If only a small proportion of subjects bearing a certain SNP display a harmful unbalance of alternative/reference expression, the threshold for significant association in GWAS studies which only measures SNP genotype will overlook the predictive value of these SNPs. Further, for many SNP in heterozygous individuals examined here, the presence of the SNP is associated with overexpression of the reference allele and not the alternative allele, and a specific negative threshold of the ratio of variant allele expression can predict disease. This relationship is not captured by current biomarker assays which measure the genetic dose of a SNP (WT, homo, het), or expression of only mutant SNP alleles.

Differences in the distributions of DAE ratios between cases and the general population indicate the presence of a regulatory site, or altered regulatory site function, which is associated with cancer susceptibility. This difference in allelic expression corresponds to a calculation of effect size, and may be determined by several methods including, but not restricted to, Cohen's d, Hedges' g, Cliff's delta, mean difference and median difference. A major benefit of DAE case-control association studies is that the target gene(s) and function of the discovered susceptibility locus is known from the outset of the study: regulation of transcript levels. Mapping of the actual functional variant (cis-variant) is facilitated by the growing knowledge of genetic variation.

The inventors overcome this by searching for differential expression of either a reference allele, or an alternative allele of several SNPs located in the same region, or risk locus as a “risk-lead SNP” identified by GWAS, to find candidates for predictive assays which measure mRNA expression level of the R and A allele. The inventors develop a method to measure AL of such risk SNP to predict an individual's risk for disease, by analysis differential allele expression of variant alleles relative to their respective wildtype allele at one, or a plurality of sites of heterozygous polymorphisms (e.g. in known 17q22 risk locus, and a new CDC16 gene ENSG00000130177 containing SNP rs3211416).

This work has revealed the power of integrating allelic expression data for the purpose of determining a subject's risk of developing cancer. By inspecting the distribution of AE ratios in normal tissue samples—breast and blood—, the inventors present a novel approach to identifying risk—case-control association analysis using AE ratios, which proved robust when multiple cis-regulatory variants are involved in a complex risk genetic structure. Finally, the estimated effect sizes are large (detected in rs2628315) to medium (detected in rs17817901) and are independent of the sample size, unlike p-values. This evidence that AE is associated with risk, is a direct indication of cis-regulatory variants control. Transcribed variants in all three genes show differential allelic expression and are in strong to complete LD with the lead-variant for risk in this locus; notably, all aeSNPs with equimolar allelic expression were in weak to no LD with the risk lead-variant. Importantly, there is a direct association between differential allelic expression and disease risk.

The risk association found for AE ratios measured in breast tissue were also valid in blood, setting greatly facilitates the translation of these results to a clinical setting. A similar profile of association of AE ratios was measured at rs2628315 and rs17817901; the effects were similar in direction and size.

The approach of the invention, tailors the threshold of each SNP to take into account variability in distribution of different gene expression levels, using the effect size using the Hedges' g, a standardized mean difference method, which is independent of the sample size. Increasing the sample size may shrink the 95% CI of the estimated effect size, but the threshold for different AE should remain relevant. Only individuals heterozygous for the transcribed variants used for quantification are informative, however, compared to the classical association studies using genotype frequencies for individual SNPs, besides the increased statistical power of using a quantitative phenotype, AE ratios report simultaneously on the effect of all the variants regulating a gene. All SNP presented as biomarkers for cancer susceptibility in the present study, are found in more than 1% of the general population (Table. 3).

### Example 2: Genetic Screening Workflow According to the Invention Using qPCR

A subject with a familial history of breast cancer is offered genetic screening in order to determine whether they have a higher risk than the general population of developing breast cancer. The subject provides a blood sample, from which mRNA is isolated, then reverse transcribed into cDNA. Quantitative real time PCR using allele-specific primers is used to measure the mRNA expression level of each allele of the risk SNPs rs17817901 and rs2628315. The primer pairs for each risk allele are also added to a sample containing buffer only, in order to determine the background signal for each assay. TaqMan genotyping assays were used to perform qPCR specific for rs17817901 and a rs2628315 assays, which are quantified relative to a standard curve obtained from the CEPH cell lines expressing each SNP.

rs17817901:

Sample reference allele A primers: 5
a. Background for reference allele A primers: 1
b. Reference allele expression above background: 5−1=4

Sample alternative allele G primers: 5
a. Background for alternative allele G primers: 3
b. Alternative allele expression above background: 5−3=2

AE Threshold: log₂(Alternative A/Reference G): log 2(2/4)=−1
a. Threshold for presence of DAE of rs17817901 in blood: −0.737
b. Subject DAE −1<−0.737 rs17817901 threshold
   rs2628315:

Reference allele G: 8
a. Background for reference allele G: 1
b. Reference allele expression above background: 8−1=7

Alternative allele A: 1
a. Background for alternative allele A: 1
b. Alternative allele expression above background 1−1=0

The subject is not heterozygous for the alternative rs2628315 allele, as the expression of the variant allele A was equivalent to that of a background sample for the risk SNP alternative allele A primer pair. The assay determines that the patient is heterozygous for the risk allele rs17817901, as the CT of the variant allele G is above that of a background sample. Further, the subject exhibits DAE of the risk SNP rs17817901, with preferred expression of the reference allele in comparison to the variant allele. The subject is offered genetic counselling, and is advised to seek yearly breast cancer screening.

### Example 3: Genetic Screening Workflow According to the Invention Using mRNA Sequencing

A subject with a familial history of breast cancer is offered genetic screening in order to determine whether they have a higher risk than the general population of developing breast cancer. The subject provides a blood sample, from which mRNA is isolated, and prepared for mRNA sequencing using a standard Illumina workflow. The resulting mRNA sequencing number of reads per million (RPM) for both alleles of the rs17817901 and rs2628315 locus:

| rs17817901: | rs2628315: |
|---|---|
| Reference allele A: 200 RPM | Reference allele G: 300 RPM |
| Variant allele G: 0 RPM | Variant allele A: 0 RPM |

The assay determines that the patient is not characterised by the presence of a variant risk allele rs17817901 or rs2628315, as neither is expressed above the level of a background sample. As a result, the subject is informed that the assay has not demonstrated an above average risk of developing malignant breast cancer in their lifetime compared to the general population.

### Example 4: Method of Treating Preventively an Individual Having a Breast Tissue Sample, Normal or Tumoural, Classified as High-Risk by the Method According to the Invention

The invention further related to a method of preventively treating an individual with high-risk of developing breast cancer, and is particularly useful when the malignancy or tumour growth is highly probable.

According to the method, a subject having indication for risk screening for breast cancer, provides a blood sample, from which mRNA is isolated, then reverse transcribed into cDNA as specified in example 2, or prepared for mRNA sequencing as in Example 3. In some embodiments, the AR ratio for is determined, rs17817901 or rs12936860, and according to the formula log 2(Alt/Ref), the patient is assigned a risk of malignant breast cancer development if the AE threshold for rs17817901 or rs12936860 is within the range of −1.6 to 0.18. In particular embodiments, the threshold below which a risk is assigned to a subject is about 0.-7. In other particular embodiments, where an AE ratio is determined for SNP rs2628315, and the sample is a blood sample, the AE threshold is within the range of −2.3 to −0.7. in more particular embodiments the for this SNP threshold is about −1.4.

In alternative embodiments of the method according to the invention relating to assigning a probable diagnosis of malignant breast cancer the sample comprises, or essentially consists of breast tissue, for example, biopsy tissue. In more particular embodiments, the sample is breast tissue, and the AE ratio is the log 2(Alt/Ref), and the AE threshold applied to rs17817901 and/or rs12936860 is within the range of −1 to −0.1. In still more particular embodiments, the threshold for either of these risk SNP is about −0.5. In other particular embodiments, where one or more of the risk SNP is rs2628315, and the sample is a breast tissue sample, the AE threshold is within the range of −2 to −0.27. In more particular such embodiments, the threshold is about −1.2.

In some embodiments, the patient assigned a status of highly malignant tumour is treated with an antineoplastic drug suitable for highly malignant breast cancer neoplasms selected from the list consisting of
an anthracycline-type antineoplastic drug, particularly an anthracycline-type antineoplastic drug selected from daunorubicin, doxorubicin, epirubicine, and idarubicin;
a mitotic inhibitor-type antineoplastic drug, particularly a mitotic inhibitor-type antineoplastic drug selected from capazitaxel, docetaxel, nab-paclitaxel, paclitaxel, vinblastine, vincristine, and vinorelbine;
an antineoplastic platinum complex, particularly an antineoplastic platinum complex selected from carboplatin, satraplatin, cisplatin, dicycloplatin, nedaplatin, oxaliplatin, picoplatin, triplatin and tetranitrate;
an alkylating antineoplastic drug, particularly an alkylating antineoplastic drug selected from altretamine, bendamustine, busulfan, carboplatin, carmustine, cisplatin, cyclophosphamide, chlorambucil, dacarbayine, ifosfamide, lomustine, mechlorethamine, melphalan, oxaliplatin, temozolomide, thiptepa, and trabectedin; and/or
an antimetabolite-type antineoplastic drug, particularly an antimetabolite-type antineoplastic drug selected from; azacytidine, 5-fluorouracil, 6-mercaptopurine, capecitabine, clofarabine, cytarabine, decitabine, floxuridine, fludarabine, gemcitabine, hydroxyurea, hydroxycarbamide, methotrexate, nelarabine, pemetrexed, pentostatin, pralatrexate, and phototrexate.

In some embodiments a parallel analysis of a breast tissue sample is undertaken, the tissue is examined for common phenotype markers that can inform the appropriate course for patient treatment, by genetic sequencing or histopathological analysis.

In some embodiments the tumour sample expresses HER2, and a pharmaceutical formulation comprising a HER2 targeting antineoplastic drug is provided to the patient, particularly a HER2 targeting antineoplastic drug selected from trastuzumab, pertuzumab, SYD985, RC48, A166, HER2ALT-P7, T-DM1, ARX788, KN026, BVAC-B, MT-5111, AVX901, TAS0728, MP0274, MM-302, FS102, H2NVAC, and HER2.taNK cells, HER2-pulsed dendritic cells, or HER2-targeting T cells.

In some embodiments, the tumour sample has a PIK3CA mutation, and a pharmaceutical formulation comprising pathway inhibitors is provided to the patient, particularly alpelisib, inavolisib, buparlisib, gedatolisib, pictilisib, or copanlisib.

In some embodiments, the tumour sample expresses the EGFR, and the patient is treated with a pharmaceutical formulation comprising an inhibitor of EGFR bioactivity antineoplastic drug, particularly an inhibitor of EGFR bioactivity antineoplastic drug selected from gefetinib, erlotinib, lapatinib, cetuxumib, neratinib, osimeratib, panitumamib, vandetanib, necitumumab, and dacomitinib.

In some embodiments, the tumour sample expresses the estrogen receptor (ER), and the patient is treated with a pharmaceutical formulation comprising”
a SERM antineoplastic drug, more particularly a SERM drug selected from raloxifene, toremifene or tamoxifen;
a SERD antineoplastic drug, more particularly fulvestrant, brilandestrant and elacestrant; and/or
an aromatase inhibitor antineoplastic drug, particularly an aromatase inhibitor antineoplastic drug selected from exemestane, letrozole, vorozole, formestane, fadrozole and anastrozole.

TABLE 1

| | | | | | LD with | | | | |
|---|---|---|---|---|---|---|---|---|---|
| | | Reference | Alternative | | rs2787486 | DAE - One-Sample t-Test | | | |
| rsID | Gene | Allele | Allele | MAF | (r²) | p. adjust | Mean | CIinf | CIsup |
| rs9899602 | TOM1L1 | T | C | 0.29 | 0.66 | 4.14E−06 | 0.410 | 0.298 | 0.522 |
| rs9303360 | TOM1L1 | C | T | 0.42 | 0.25 | 6.05E−01 | −0.334 | −0.629 | −0.040 |
| rs12165058 | TOM1L1 | C | T | 0.46 | 0.22 | 1.00E+00 | 0.015 | −0.089 | 0.119 |
| rs12944690 | TOM1L1/ | G | A | 0.46 | 0.20 | 9.26E−04 | −0.199 | −0.284 | −0.115 |
| | COX11 | | | | | | | | |
| rs12936860 | TOM1L1/ | G | A | 0.27 | 0.74 | 1.53E−03 | −0.209 | −0.295 | −0.122 |
| | COX11 | | | | | | | | |
| rs17817865 | TOM1L1/ | G | A | 0.27 | 0.14 | 2.68E−12 | 0.350 | 0.296 | 0.404 |
| | COX11 | | | | | | | | |
| rs2287136 | TOM1L1/ | G | A | 0.27 | 0.14 | 1.40E−01 | 0.098 | 0.030 | 0.166 |
| | COX11 | | | | | | | | |
| rs17817901 | TOM1L1/ | A | G | 0.27 | 0.74 | 3.65E−21 | −1.050 | −1.110 | −0.995 |
| | COX11 | | | | | | | | |
| rs7643 | TOM1L1/ | A | G | 0.46 | 0.22 | 3.76E−25 | −1.110 | −1.170 | −1.050 |
| | COX11 | | | | | | | | |
| rs2541240 | COX11 | G | A | 0.33 | 0.15 | 9.26E−02 | −0.359 | −0.594 | −0.124 |
| rs17817950 | COX11/ | G | A | 0.27 | 0.18 | 7.35E−01 | 0.277 | 0.024 | 0.531 |
| | STXBP4 | | | | | | | | |
| rs12947685 | COX11/ | T | C | 0.27 | 0.18 | 1.00E+00 | 0.131 | −0.047 | 0.309 |
| | STXBP4 | | | | | | | | |
| rs8064882 | COX11/ | C | T | 0.27 | 0.18 | 8.27E−01 | 0.304 | 0.019 | 0.589 |
| | STXBP4 | | | | | | | | |
| rs4794549 | COX11/ | C | A | 0.29 | 0.20 | 3.94E−02 | 0.282 | 0.116 | 0.449 |
| | STXBP4 | | | | | | | | |
| rs1420526 | STXBP4 | T | C | 0.26 | 0.00 | 1.96E−05 | 0.387 | 0.259 | 0.514 |
| rs17818238 | STXBP4 | G | A | 0.21 | 0.12 | 1.00E+00 | −0.120 | −0.280 | 0.409 |
| rs8069999 | STXBP4 | G | A | 0.19 | 0.12 | 3.45E−02 | −0.159 | −0.249 | −0.069 |
| rs2628318 | STXBP4 | G | A | 0.42 | 0.27 | 3.72E−02 | 0.227 | 0.095 | 0.358 |
| rs2628315 | STXBP4 | G | A | 0.28 | 1.00 | 1.14E−06 | 0.617 | 0.464 | 0.770 |
| rs244303 | STXBP4 | A | G | 0.41 | 0.26 | 1.00E+00 | 0.028 | −0.050 | 0.105 |
| rs244301 | STXBP4 | T | C | 0.41 | 0.26 | 4.31E−02 | −0.115 | −0.183 | −0.046 |
| rs244298 | STXBP4 | T | C | 0.41 | 0.26 | 1.63E−07 | −0.486 | −0.607 | −0.365 |

TABLE 2

| | | Controls | Cases | Hedges' | | | | Pvalue |
|---|---|---|---|---|---|---|---|---|
| | rsID | (N) | (N) | g | CI 90% | CI 95% | CI 98% | perm |
| Breast | rs17817901 | 23 | 50 | −0.486 | [−0.810; | [−0.889; | [−0.970; | 0.0534 |
| Tissue | | | | | −0.206] | −0.147] | −0.086] | |
| | rs2628315 | 19 | 42 | −1.237 | [−1.811; | [−1.928; | [−2.052; | 0.0002 |
| | | | | | −0.522] | −0.366] | −0.217] | |
| Blood | rs17817901 | 14 | 24 | −0.737 | [−1.314; | [−1.431; | [−1.556; | 0.034 |
| | | | | | −0.120] | −0.002] | 0.175] | |
| | rs2628315 | 12 | 23 | −1.419 | [−2.007; | [−2.156; | [−2.339; | 0 |
| | | | | | −0.873] | −0.780] | −0.696] | |

TABLE 3

| chr | pos_hg38 | rsID | ref | alt | AFR | AMR | ASN | EUR |
|---|---|---|---|---|---|---|---|---|
| 17 | 54902624 | rs9914088 | T | C | 0.19 | 0.22 | 0.23 | 0.31 |
| 17 | 54904492 | rs12949718 | C | T | 0.28 | 0.24 | 0.26 | 0.32 |
| 17 | 54905855 | rs9908752 | C | T | 0.24 | 0.22 | 0.23 | 0.31 |
| 17 | 54907524 | rs9895901 | A | G | 0.18 | 0.21 | 0.23 | 0.31 |
| 17 | 54907990 | rs9897646 | G | C | 0.17 | 0.19 | 0.12 | 0.29 |
| 17 | 54908621 | rs9904377 | G | A | 0.21 | 0.22 | 0.23 | 0.31 |
| 17 | 54909484 | rs9899545 | T | C | 0.18 | 0.19 | 0.12 | 0.29 |
| 17 | 54909547 | rs9899602 | T | C | 0.18 | 0.19 | 0.12 | 0.29 |
| 17 | 54910184 | rs2877634 | A | G | 0.37 | 0.25 | 0.26 | 0.31 |
| 17 | 54910871 | rs17745123 | G | T | 0.16 | 0.19 | 0.12 | 0.29 |
| 17 | 54911710 | rs9913784 | A | T | 0.21 | 0.21 | 0.23 | 0.31 |
| 17 | 54912237 | rs28564882 | C | T | 0.18 | 0.21 | 0.15 | 0.3 |
| 17 | 54914907 | rs9910653 | G | A | 0.2 | 0.22 | 0.22 | 0.32 |
| 17 | 54916595 | rs66898273 | C | T | 0.18 | 0.21 | 0.12 | 0.3 |
| 17 | 54916813 | rs12602751 | C | A | 0.2 | 0.22 | 0.22 | 0.32 |
| 17 | 54916849 | rs35079296 | T | G | 0.18 | 0.21 | 0.12 | 0.29 |
| 17 | 54918303 | rs7216138 | G | A | 0.2 | 0.22 | 0.22 | 0.32 |
| 17 | 54918789 | rs1962045 | C | T | 0.18 | 0.21 | 0.12 | 0.3 |
| 17 | 54919190 | rs9914596 | C | T | 0.18 | 0.21 | 0.12 | 0.3 |
| 17 | 54920514 | rs9914836 | C | G | 0.17 | 0.19 | 0.09 | 0.29 |
| 17 | 54920807 | rs9915913 | G | T | 0.33 | 0.22 | 0.12 | 0.3 |
| 17 | 54920956 | rs71361737 | G | GAC | 0.37 | 0.22 | 0.12 | 0.3 |
| 17 | 54926783 | rs6504947 | T | A | 0.79 | 0.48 | 0.37 | 0.56 |
| 17 | 54928355 | rs12952253 | A | G | 0.05 | 0.18 | 0.23 | 0.3 |
| 17 | 54928963 | rs7209926 | A | G | 0.23 | 0.2 | 0.23 | 0.3 |
| 17 | 54928978 | rs7208123 | G | A | 0.06 | 0.18 | 0.23 | 0.3 |
| 17 | 54929529 | rs9916547 | G | A | 0.23 | 0.2 | 0.23 | 0.3 |
| 17 | 54930710 | rs35647022 | C | T | 0.05 | 0.16 | 0.09 | 0.28 |
| 17 | 54931492 | rs8065361 | A | T | 0.23 | 0.2 | 0.23 | 0.3 |
| 17 | 54931919 | rs72829484 | G | T | 0.06 | 0.18 | 0.23 | 0.3 |
| 17 | 54932610 | rs9912589 | G | A | 0.23 | 0.2 | 0.23 | 0.3 |
| 17 | 54932737 | rs12603899 | T | C | 0.23 | 0.2 | 0.23 | 0.3 |
| 17 | 54933309 | rs12949538 | T | | 0.05 | 0.16 | 0.12 | 0.28 |
| 17 | 54933394 | rs9891704 | G | | 0.23 | 0.2 | 0.23 | 0.3 |
| 17 | 54934696 | rs9900816 | A | | 0.24 | 0.2 | 0.23 | 0.3 |
| 17 | 54935566 | rs9907961 | G | | 0.24 | 0.2 | 0.23 | 0.3 |
| 17 | 54935928 | rs9909096 | T | | 0.24 | 0.2 | 0.23 | 0.3 |
| 17 | 54935996 | rs35465233 | A | | 0.05 | 0.18 | 0.23 | 0.3 |
| 17 | 54936006 | rs34652394 | T | | 0.05 | 0.18 | 0.22 | 0.3 |
| 17 | 54936234 | rs17745183 | T | | 0.05 | 0.18 | 0.23 | 0.3 |
| 17 | 54936619 | rs17745189 | G | | 0.05 | 0.16 | 0.12 | 0.28 |
| 17 | 54938769 | rs12937760 | T | | 0.05 | 0.17 | 0.12 | 0.28 |
| 17 | 54938794 | rs35297544 | C | | 0.05 | 0.16 | 0.12 | 0.28 |
| 17 | 54940386 | rs56175787 | G | | 0.05 | 0.16 | 0.12 | 0.28 |
| 17 | 54940920 | rs17817847 | A | | 0.05 | 0.16 | 0.12 | 0.28 |
| 17 | 54944562 | rs35097996 | C | | 0.05 | 0.16 | 0.12 | 0.28 |
| 17 | 54948114 | rs8066833 | G | | 0.05 | 0.16 | 0.12 | 0.28 |
| 17 | 54948328 | rs8066588 | T | | 0.05 | 0.16 | 0.12 | 0.28 |
| 17 | 54949462 | rs71159274 | T | | 0.05 | 0.16 | 0.12 | 0.28 |
| 17 | 54951254 | rs8073158 | A | | 0.05 | 0.17 | 0.12 | 0.28 |
| 17 | 54951967 | rs3087650 | A | | 0.05 | 0.16 | 0.12 | 0.28 |
| 17 | 54952865 | rs12936860 | A | | 0.05 | 0.16 | 0.12 | 0.28 |
| 17 | 54953100 | rs12937006 | T | | 0.05 | 0.16 | 0.12 | 0.28 |
| 17 | 54957267 | rs17817877 | A | | 0.05 | 0.15 | 0.09 | 0.28 |
| 17 | 54958067 | rs8076984 | C | | 0.05 | 0.16 | 0.12 | 0.28 |
| 17 | 54958133 | rs35726440 | G | | 0.04 | 0.16 | 0.11 | 0.27 |
| 17 | 54959168 | rs17745231 | G | | 0.05 | 0.16 | 0.12 | 0.28 |
| 17 | 54959998 | rs12938118 | C | | 0.05 | 0.16 | 0.12 | 0.28 |
| 17 | 54961293 | rs1802212 | C | | 0.05 | 0.16 | 0.12 | 0.28 |
| 17 | 54961384 | rs17817901 | G | | 0.05 | 0.16 | 0.12 | 0.28 |
| 17 | 54964159 | rs12150038 | G | | 0.05 | 0.16 | 0.12 | 0.28 |
| 17 | 54964529 | rs201764136 | T | | 0.04 | 0.16 | 0.12 | 0.27 |
| 17 | 54964531 | rs141544971 | C | | 0.04 | 0.16 | 0.12 | 0.27 |
| 17 | 54964604 | rs12951542 | C | | 0.05 | 0.16 | 0.12 | 0.28 |
| 17 | 54966443 | rs72831295 | C | | 0.05 | 0.17 | 0.12 | 0.28 |
| 17 | 54969776 | rs9915183 | A | | 0.29 | 0.17 | 0.09 | 0.27 |
| 17 | 54970138 | rs7222197 | A | | 0.29 | 0.17 | 0.09 | 0.27 |
| 17 | 54970958 | rs17745344 | G | | 0.03 | 0.14 | 0.09 | 0.26 |
| 17 | 54971081 | rs9895808 | G | | 0.29 | 0.17 | 0.09 | 0.27 |
| 17 | 54971108 | rs9897447 | C | | 0.29 | 0.17 | 0.09 | 0.27 |
| 17 | 54971181 | rs9896044 | G | | 0.28 | 0.17 | 0.09 | 0.27 |
| 17 | 54971563 | rs9902687 | A | | 0.29 | 0.17 | 0.09 | 0.27 |
| 17 | 54972508 | rs8080491 | A | | 0.3 | 0.17 | 0.09 | 0.27 |
| 17 | 54972626 | rs6504948 | C | | 0.29 | 0.17 | 0.09 | 0.27 |
| 17 | 54972772 | rs6504949 | G | | 0.29 | 0.17 | 0.09 | 0.27 |
| 17 | 54975207 | rs35051208 | T | | 0.05 | 0.15 | 0.09 | 0.27 |
| 17 | 54976018 | rs8078550 | G | | 0.29 | 0.17 | 0.09 | 0.27 |
| 17 | 54977006 | rs9914732 | G | | 0.29 | 0.17 | 0.09 | 0.27 |
| 17 | 54977136 | rs9916642 | C | | 0.29 | 0.17 | 0.09 | 0.27 |
| 17 | 54977336 | rs9915832 | A | | 0.29 | 0.17 | 0.09 | 0.27 |
| 17 | 54977388 | rs9893306 | T | | 0.29 | 0.17 | 0.09 | 0.27 |
| 17 | 54977885 | rs9894529 | T | | 0.28 | 0.17 | 0.09 | 0.27 |
| 17 | 54979110 | rs6504950 | A | | 0.3 | 0.17 | 0.09 | 0.27 |
| 17 | 54979614 | rs6504951 | G | | 0.29 | 0.17 | 0.09 | 0.27 |
| 17 | 54980030 | rs71300611 | CTA | | 0.3 | 0.17 | 0.09 | 0.27 |
| 17 | 54980386 | rs9903146 | T | | 0.29 | 0.17 | 0.09 | 0.27 |
| 17 | 54980403 | rs9902950 | T | | 0.28 | 0.17 | 0.09 | 0.27 |
| 17 | 54980504 | rs9903220 | G | | 0.29 | 0.17 | 0.09 | 0.27 |
| 17 | 54980532 | rs9903444 | T | | 0.3 | 0.17 | 0.09 | 0.27 |
| 17 | 54980553 | rs9903825 | A | | 0.29 | 0.17 | 0.09 | 0.27 |
| 17 | 54981315 | rs28558726 | G | | 0.29 | 0.17 | 0.09 | 0.27 |
| 17 | 54981446 | rs16955471 | T | | 0.29 | 0.17 | 0.09 | 0.27 |
| 17 | 54981646 | rs7221223 | G | | 0.06 | 0.15 | 0.09 | 0.26 |
| 17 | 54982414 | rs9892848 | A | | 0.27 | 0.16 | 0.08 | 0.24 |
| 17 | 54982415 | rs9890971 | T | | NA | NA | NA | NA |
| 17 | 54982672 | rs9891865 | T | | 0.29 | 0.17 | 0.09 | 0.27 |
| 17 | 54982853 | rs9892173 | T | | 0.29 | 0.18 | 0.09 | 0.27 |
| 17 | 54982968 | rs9892976 | A | | 0.29 | 0.17 | 0.09 | 0.26 |
| 17 | 54983714 | rs1990674 | A | | 0.29 | 0.17 | 0.09 | 0.27 |
| 17 | 54984261 | rs9902718 | C | | 0.29 | 0.17 | 0.09 | 0.27 |
| 17 | 54985394 | rs17818028 | G | | 0.05 | 0.15 | 0.09 | 0.26 |
| 17 | 54985542 | rs10468513 | A | | 0.29 | 0.17 | 0.09 | 0.27 |
| 17 | 54987189 | rs56348638 | T | | 0.29 | 0.17 | 0.09 | 0.27 |
| 17 | 54988446 | rs7219874 | T | | 0.29 | 0.17 | 0.09 | 0.27 |
| 17 | 54990322 | rs12940106 | G | | 0.05 | 0.15 | 0.09 | 0.26 |
| 17 | 54990632 | rs8082471 | A | | 0.29 | 0.17 | 0.09 | 0.27 |

1. A method to treat breast cancer in an individual, said method comprising:
a. determining the genotype for each of one or more risk single nucleotide polymorphisms (SNP) in a sample obtained from the subject, particularly a blood sample,
wherein the one or more risk SNP are selected from the list consisting of selected from:
rs17817901, rs12936860, and/or rs2628315; or
a SNP in linkage disequilibrium with a risk SNP selected from rs17817901, rs12936860, and/or rs2628315,
wherein linkage disequilibrium is defined by a Pearson coefficient of correlation (r²) of at least (>) 0.2, more particularly an r² value >0.6; and
b. in a measurement step, if the subject is heterozygous for a reference allele and an alternative allele, determining for said one or more SNP,
an mRNA expression level of a reference allele, and
an mRNA expression level of the alternative allele,
to provide a reference allele expression level and an alternative allele expression level; and
c. administering to the individual an effect amount of an antineoplastic drug if for at least one of said one or more risk SNP:
the reference allele expression level is significantly more than (>) the alternative allele expression level,
wherein for the risk SNP the log₂(mean alternative allele expression levels/mean reference allele expression level) of a cohort of healthy controls, is >log₂(mean alternative allele expression levels/mean reference allele expression level) of a cohort of patients diagnosed with breast cancer, or
the alternative allele expression level is significantly >the reference allele expression level,
wherein for the risk SNP the log₂(mean alternative allele expression levels/mean reference allele expression level) of a cohort of healthy controls, is <log₂(mean alternative allele expression levels/mean reference allele expression level) of a cohort of patients diagnosed with breast cancer.

2. The method according to claim 1, wherein each of the one or more risk SNP is located within a transcribed region of a gene selected from STXBP4, COX11 and/or TOM1L1.

3. The method according to claim 1, wherein each of the one or more risk SNP is present in the general population at a frequency of at least (≥) 1%.

4. The method according to claim 1, wherein the one or more risk SNP comprises, or is rs17817901;
wherein the individual is administered an effective amount of the antineoplastic drug if the mRNA expression level of a reference rs17817901 adenine (A) allele is ≥about 2-fold the mRNA expression level of an alternative rs1781790 guanine (G) allele.

5. The method according to claim 1, wherein the one or more risk SNP comprises or is rs12936860;
wherein the individual is administered an effective amount of the antineoplastic drug if the mRNA expression level of a reference rs12936860 G allele is ≥about 2-fold the mRNA expression level of an alternative of rs12936860 A allele.

6. The method according to claim 1, wherein the one or more risk SNP comprises or is rs2628315;
wherein the individual is administered an effective amount of the antineoplastic drug if the mRNA expression level of a reference rs2628315 G allele is ≥about 1.5-fold the mRNA expression level of an alternative rs2628315 A allele.

7. The method according to claim 1, wherein in the measurement step, the mRNA expression level is determined for:
rs17817901, and/or rs12936860, and
rs2628315.

8. The method according to claim 1, wherein the sample is a blood sample.

9. The method according to claim 1, wherein the sample comprises, or essentially consists of breast tissue.

10. The method according to claim 1, wherein the measurement step is followed by:
a calculation step, wherein an allelic expression (AE) ratio is determined for each of the one or more risk SNPs,
wherein the AE ratio is obtained using the formula:
log₂(alternative allele expression level/reference allele expression level); and
wherein the individual is administered an effective amount of the antineoplastic drug if for at least one of one or more risk SNP:
the AE ratio is ≤an AE threshold,
wherein the mean AE ratio as specified in a. of a cohort of healthy controls is >the mean AE ratio of a cohort of patients diagnosed with breast cancer, or
the AE ratio is ≥the AE threshold,
wherein the mean AE ratio of a cohort of healthy controls is <the mean AE ratio of a cohort of patients diagnosed with breast cancer.

11. The method according to claim 10, wherein the AE threshold for the one or more risk SNP is a normalised measure of the effect size comparing the difference between:
the mean AE ratio obtained from samples of a cohort of ≥about 10 patients characterised as heterozygous for said risk SNP, and each patient having been diagnosed with breast cancer, and
the mean AE ratio obtained from samples from a cohort of ≥about 10 healthy subjects heterozygous for said risk SNP;
particularly wherein the normalised measure of effect size is a measure selected from a Mann Whitney, Cohen's D, or Hedge's g test.

12. The method according to claim 10, wherein the sample is a blood sample, and wherein the AE threshold for:
rs17817901 or rs12936860 is within the range of −1.6 to 0.18, particularly wherein the threshold is about −0.7, and/or
rs2628315 is within the range of −2.3 to −0.7, particularly wherein the threshold is about −1.4.

13. The method according to claim 10, wherein the sample comprises, or essentially consists of breast tissue;
wherein the AE threshold is
rs17817901 or rs12936860 is within the range of −1 to −0.1, particularly wherein the threshold is about −0.5, and/or
rs2628315 is within the range of −2 to −0.2, particularly wherein the threshold is about −1.2.

14. The method according to claim 1, wherein the mRNA expression levels are obtained by allele-specific, quantitative, mRNA measurement methodology, particularly a methodology selected from mRNA sequencing, microarray, or real-time quantitative polymerase chain reaction.

15. The method according to claim 1, wherein the antineoplastic drug is selected from the list consisting of:
an anthracycline-type antineoplastic drug, particularly an anthracycline-type antineoplastic drug selected from daunorubicin, doxorubicin, epirubicine, and idarubicin;
a mitotic inhibitor-type antineoplastic drug, particularly a mitotic inhibitor-type antineoplastic drug selected from capazitaxel, docetaxel, nab-paclitaxel, paclitaxel, vinblastine, vincristine, and vinorelbine;
an antineoplastic platinum complex, particularly an antineoplastic platinum complex selected from carboplatin, satraplatin, cisplatin, dicycloplatin, nedaplatin, oxaliplatin, picoplatin, triplatin and tetranitrate;
an alkylating antineoplastic drug, particularly an alkylating antineoplastic drug selected from altretamine, bendamustine, busulfan, carboplatin, carmustine, cisplatin, cyclophosphamide, chlorambucil, dacarbayine, ifosfamide, lomustine, mechlorethamine, melphalan, oxaliplatin, temozolomide, thiptepa, and trabectedin; and/or
an antimetabolite-type antineoplastic drug, particularly an antimetabolite-type antineoplastic drug selected from; azacytidine, 5-fluorouracil, 6-mercaptopurine, capecitabine, clofarabine, cytarabine, decitabine, floxuridine, fludarabine, gemcitabine, hydroxyurea, hydroxycarbamide, methotrexate, nelarabine, pemetrexed, pentostatin, pralatrexate, and phototrexate;
a HER2 targeting antineoplastic drug, particularly a HER2 targeting antineoplastic drug selected from trastuzumab, pertuzumab, SYD985, RC48, A166, HER2ALT-P7, T-DM1, ARX788, KN026, BVAC-B, MT-5111, AVX901, TAS0728, MP0274, MM-302, FS102, H2NVAC, HER2.taNK cells, HER2-pulsed dendritic cells, and HER2-targeting T cells;
a PI3KCA pathway inhibitor drug, particularly a PI3KCA pathway inhibitor drug selected from alpelisib, inavolisib, buparlisib, gedatolisib, pictilisib, or copanlisib;
a selective oestrogen receptor modulator (SERM) antineoplastic drug, particularly a SERM drug selected from raloxifene, toremifene and tamoxifen;
a selective estrogen receptor degraders (SERD) antineoplastic drug, particularly a SERD selected from fulvestrant, brilandestrant and elacestrant;
an aromatase inhibitor antineoplastic drug, particularly an aromatase inhibitor antineoplastic drug selected from exemestane, letrozole, vorozole, formestane, fadrozole and anastrozole;
an inhibitor of EGFR bioactivity antineoplastic drug, particularly an inhibitor of EGFR bioactivity antineoplastic drug selected from gefetinib, erlotinib, lapatinib, cetuxumib, neratinib, osimeratib, panitumamib, vandetanib, necitumumab, and dacomitinib.
